(19) United States
(12) Patent Application Publication
(10) Pub. No.: US 2007/0122823 A1
(43) Pub. Date: May 31, 2007

(54) Amniotic fluid cell-free fetal DNA fragment size pattern for prenatal diagnosis

(76) Inventors: Diana W. Bianchi, Brookline, MA (US); Kirby L. Johnson, Quincy, MA (US); Olav Lapaire, Riehen (CH)

Correspondence Address: CHOATE, HALL & STEWART LLP, TWO INTERNATIONAL PLACE, BOSTON, MA, 02110, US

(21) Appl. No.: 11/513,797

(22) Filed: Aug. 31, 2006

Related U.S. Application Data
(60) Provisional application No. 60/713,540, filed on Sep. 1, 2005.

(51) Int. Cl. C12Q 1/68; G06F 19/00

(52) U.S. Cl. 435/6; 702/20

(57) ABSTRACT

The present invention relates to improved methods of prenatal diagnosis, screening, monitoring and/or testing. The inventive methods include analysis of the fragment size distribution of cell-free fetal DNA isolated from amniotic fluid. The inventive methods allow for rapid screening of fetal characteristics such as chromosomal abnormalities and for prenatal diagnosis of a variety of diseases and conditions. Since the new methods do not require cell culture, they can be performed more rapidly than conventional fetal karyotypes.

### RELATED APPLICATIONS

This application claims priority from Provisional Patent Application No. 60/713,540, filed Sep. 1, 2005 and entitled “Amniotic Fluid Cell-Free Fetal DNA Fragment Size Pattern for Prenatal Diagnosis”. The Provisional Application is incorporated herein by reference in its entirety. The present application is also related to U.S. Application Ser. No. 10/577,341 filed Apr. 28, 2006, which is a U.S. National Phase Application under 35 U.S.C § 371 of International Application PCT/US04/035929 (published PCT application No. WO 2005/044086) filed Oct. 29, 2004, which itself claims priority from Provisional Application No. 60/515,735 filed Oct. 30, 2003. Each of these applications is incorporated herein by reference in its entirety.

### GOVERNMENT INTERESTS

The work described herein was funded by the National Institutes of Health (Grant No. NIH HD42053). The United States government may have certain rights in the invention.

### BACKGROUND OF THE INVENTION

Genetic disorders and congenital abnormalities (also called birth defects) occur in about 3 to 5% of all live births (A. Robinson and M. G. Linden, “Clinical Genetic Handbook”, 1993, Blackwell Scientific Publications: Boston, Mass.). Combined, genetic disorders and congenital abnormalities have been estimated to account for up to 30% of pediatric hospital admissions (C. R. Scriver et al., Can. Med. Assoc. J. 1973, 108: 1111-1115; E. W. Ling et al., Am. J. Perinatal. 1991, 8: 164-169) and to be responsible for about half of all childhood deaths in industrialized countries (R. J. Berry et al., Public Health Report, 1987, 102: 171-181; R. A. Hoekelman and I. B. Pless, Pediatrics, 1998, 82: 582-595). In the US, birth defects are the leading cause of infant mortality (R. N. Anderson et al., Month. Stat. Rep. 1997, Vol. 45, No 11, Suppl. 2, p. 55). Furthermore, genetic disorders and congenital anomalies contribute substantially to long-term disability; they are associated with enormous medical-care costs (A. Czeizel et al., Mutat. Res. 1984, 128: 73-103; Centers of Disease Control, Morb. Mortal. Weekly Rep. 1989, 38: 264-267; S. Kaplan, J. Am. Coll. Cardiol. 1991, 18: 319-320; C. Cunniff et al., Clin. Genet. 1995, 48: 17-22) and create a heavy psychological and emotional burden on those afflicted and/or their families. For these and other reasons, prenatal diagnosis has long been recognized as an essential facet of the clinical management of pregnancy itself as well as a critical step toward the detection, prevention, and, eventually, treatment of genetic disorders.

Conventional chromosome analysis methods have remained the gold standard for the prenatal exclusion of aneuploidy. Such methods are based on the selective staining of chromosomes originating from fetal cells, which results in the formation of a characteristic staining (or banding) pattern along the length of the chromosomes, allowing visualization and unambiguous identification of all the chromosomes. Examination of the karyotypes determined by these banding methods can reveal the presence of numerical and structural chromosomal abnormalities over the whole genome. Fetal cells for use in these karyotyping methods are arrested in the metaphase stage of mitosis, where the structures of the chromosomes appear most distinctly. Fetal cells are traditionally isolated from samples of amniotic fluid (obtained by amniocentesis), chorionic villi (obtained by chorionic villus sampling), or fetal blood (obtained by cordocentesis or percutaneous umbilical cord blood sampling). In addition to tissue sampling and selective staining, conventional banding methods also require cell culturing, which can take between 10 and 15 days depending on the tissue source, and preparation of high quality metaphase spreads, which is tedious, time-consuming and labor-intensive (B. Eiben et al., Am. J. Hum. Genet. 1990, 47: 656-663). Furthermore, conventional chromosome analysis methods have limited sensitivity, and their standard 450-550 band level of resolution does not allow detection of small or subtle chromosomal aberrations, such as, for example, those associated with microdeletion/microduplication syndromes.

In the past decade, the application of molecular biological techniques to conventional chromosome analysis has generated new clinical cytogenetics tools that have enhanced the spectrum of disorders that can be diagnosed prenatally. These new cytogenetics tools, which are being evaluated for their potential utility in prenatal diagnosis (I. Findlay et al., J. Assist. Preprod. Genet. 1998, 15: 266-275; A. T .A. Thein et al., Prenat. Diagn. 2000, 20: 275-280; B. Pertl et al., Mol. Hum. Reprod. 1999, 5: 1176-1179; E. Pergament et al., Prenatal. Diagn. 2000, 20: 215-230) include fluorescence in situ hybridization (or FISH) and related techniques, and quantitative fluorescence polymerase chain reactions (PCR). These techniques provide increased resolution for the elucidation of structural chromosome abnormalities that cannot be detected by conventional banding analysis, such as microdeletions and microduplications, subtle translocation, complex rearrangements involving several chromosomes or taking place in subtelomeric regions. In certain of these methods, cell culture is not required, which significantly reduces test times and labor. However, in contrast to conventional banding analysis, certain molecular cytogenetic methods such as FISH, which relies on the use of chromosome specific probes to detect chromosomal abnormalities, do not allow genome-wide screening and require at least some prior knowledge regarding the suspected chromosomal abnormality and its genomic location.

In addition to new techniques of prenatal diagnosis, new sources of fetal cells have also been explored. The discovery of intact fetal cells in the maternal circulation has excited general interest as an alternative source of fetal material samples to those obtained by invasive techniques such as amniocentesis, chorionic villus sampling, or percutaneous umbilical blood sampling. Extensive research has been done on intact fetal cells recovered from maternal blood. For example, it has been demonstrated by the Applicants that the number of circulating fetal nucleated cells is increased when the fetus is affected by trisomy 21 (D. W. Bianchi et al., Am. J. Hum. Genet. 1997, 61: 822-829, which is incorporated herein by reference in its entirety). Analysis of fetal cells isolated from maternal blood has also been shown to allow prenatal diagnosis of fetal chromosomal aneuploidies (S. Elias et al., Lancet, 1992, 340: 1033; D. W. Bianchi et al., Hum. Genet. 1992, 90: 368-370; D. Gänshirt-Ahlert et al., Am. J. Reprod. Immunol. 1993, 30: 193-200; J. L. Simpson et al., J. Am. Med. Assoc. 1993, 270: 2357-2361; F. de la Cruz et al., Fetal Diagn. Ther. 1998, 13: 380).

However, because of the scarcity of intact fetal cells in most maternal blood samples, clinical applications await further technological developments (D. W. Bianchi et al., Prenat. Diagn. 2002, 22: 609-615). Another obstacle is the probable persistence of fetal lymphocytes in the maternal circulation, resulting in “contamination” of fetal cells of interest (i.e., those originating from the current pregnancy). Although considerable progress has been made in isolation, separation and enrichment of fetal cells for analysis (J. L. Simpson and S. Elias, J. Am. Med. Assoc. 1993, 270: 2357-2361; M. C. Cheung et al., Nat. Genet. 1996, 14: 264-268; R. M. Bohmer et al., Br. J. Haematol. 1998, 103: 351-360; E. Di Naro et al., Mol. Hum. Reprod. 2000, 6: 571-574; E. Parano et al., Am. J. Med. Genet. 2001, 101: 262-267), these steps are time-consuming, labor-intensive and require expensive equipment.

In 1997, Lo and coworkers (Y. M .D. Lo et al., Lancet, 1997, 350: 485-487) demonstrated the presence of male fetal DNA sequences in the serum and plasma of pregnant women. Subsequently, this same group extended their observation by quantifying the fetal DNA in maternal plasma (Y. M .D. Lo et al., Am. J. Hum. Genet. 1998, 62: 768-775), and studying its kinetics and physiology (Y. M .D. Lo et al., Am. J. Hum. Genet. 1999, 64: 218-224). Since then, a multitude of clinical applications have been reported (B. Pertl and D. W. Bianchi, Obstet. Gynecol. 2001, 98: 483-490; Y. M .D. Lo et al., Clin. Chem. 1999, 45: 1747-1751) including the determination of fetal gender and identification of fetal rhesus D status (B. H. Faas et al., Lancet, 1998, 352: 1196; Y. M .D. Lo et al., New Engl. J. Med. 1998, 339: 1734-1738; S. Hahn et al., Ann. N.Y. Acad. Sci. 2000, 906: 148-152; X. Y. Zhong et al., Brit. J. Obstet. Gynaecol. 2000, 107: 766-769; H. Honda et al., Clin. Med. 2001, 47: 41-46; H. Honda et al., Hum. Genet. 2002, 110: 75-79). Elevated concentrations of circulating fetal DNA have been measured by real-time quantitative PCR technology in pregnancies with pre-eclampsia (Y. M .D. Lo et al., Clin. Med. 1999, 45: 184-188; T. N. Leung et al., Clin. Med. 2001, 47: 137-139; X. Y. Zhong et al., Ann. N.Y. Acad. Sci. 2001, 945: 134-180), preterm labor (T. N. Leung et al., Lancet, 1998, 352: 1904-1905), hypernemesis gravidarum (A. Sekizawa et al., Clin. Med. 2001, 47: 2164-2165), and invasive placenta (A. Sekizawa et al., Clin. Med. 2002, 48: 353-354). Similar approaches have been used to diagnose prenatal conditions such as myotonic dystrophy (P. Amicucci et al., Clin. Chem. 2000, 46: 301-302), achondroplasia (H. Saito et al., Lancet, 2000, 356: 1170), Down syndrome (Y. M .D. Lo et al., Clin. Med. 1999, 45: 1747-1751; X. Y. Zhong et al., Prenatal Diagn. 2000, 20: 795-798; L. L. Poon et al., Lancet, 2000, 356: 1819-1820), aneuploidy (C. P. Chen et al., Prenat. Diag. 2000, 20: 355-357; C. P. Chen et al., Clin. Chem. 2001, 47: 937-939), and paternally inherited cystic fibrosis (M. C. Gonzalez-Gonzalez et al., Prenatal Diagn. 2002, 22: 946-948).

Compared to the analysis of fetal cells present in maternal blood, the analysis of cell-free fetal DNA isolated from maternal plasma presents the advantage of being rapid, robust and easy to perform. In addition, the fetal DNA originates exclusively from the fetus involved in the current pregnancy. However, due to the presence of maternal DNA in the plasma, the use of cell-free fetal DNA for prenatal diagnosis is limited to paternally inherited disorders or to conditions de novo present in the fetus (i.e., resulting from mutant alleles that are distinguishable from those inherited from the mother). Therefore, it is not presently applicable to autosomal recessive disorders (D. W. Bianchi, Am. J. Hum. Genet. 1998, 62: 763-764).

Clearly, improved methods of prenatal diagnosis are still needed. In particular, timely, cost-effective and sensitive methodologies that can detect chromosomal aberrations without prior knowledge of the chromosomal regions where abnormalities may be present, are highly desirable.

### SUMMARY OF THE INVENTION

The present invention provides an improved system for analyzing a fetus' genetic information. In particular, the present invention allows for the rapid prenatal screening of certain chromosomal abnormalities. More specifically, the present invention encompasses the recognition by the Applicants that the fragment size pattern of cell-free fetal DNA isolated from amniotic fluid is different for fetuses with a normal karyotype and fetuses with a chromosomal abnormality. Furthermore, the fragment size pattern was found to be characteristic for each type of chromosomal abnormality. This “fingerprint” or “signature” fragmentation pattern can find applications in the prenatal diagnosis of a variety of diseases and conditions associated with chromosomal abnormalities.

In general, the present invention involves isolating cell-free fetal DNA from a sample of amniotic fluid, and performing a DNA fragment size distribution analysis.

More specifically, in one aspect, the present invention provides a method of prenatal diagnosis comprising steps of: providing a sample of amniotic fluid fetal DNA comprising a plurality of fetal DNA fragments having different sizes; analyzing the amniotic fluid fetal DNA to obtain a fragment size distribution pattern of the amniotic fluid fetal DNA; and based on the fragment size distribution pattern obtained, providing a prenatal diagnosis.

Preferably, the amniotic fluid fetal DNA is obtained by: providing a sample of amniotic fluid obtained from a woman pregnant with a fetus; removing cell populations from the sample of amniotic fluid to obtain a remaining amniotic fluid material; and treating the remaining amniotic material such that cell-free fetal DNA present in the remaining amniotic material is extracted and made available for analysis, resulting in amniotic fluid fetal DNA. When substantially all cell populations are removed from the sample of amniotic fluid, the amniotic fluid fetal DNA consists essentially of cell-free fetal DNA. When the remaining amniotic material comprises some cells, the amniotic fluid fetal DNA comprises cell-free fetal DNA and DNA originating from the cells present in the remaining amniotic material. The remaining material may be frozen, and stored for a period of time under suitable conditions, and later thawed prior to the treating step. Substantially all cell populations that are still present in the remaining amniotic material after the thawing step may be removed prior to the treating step.

In certain embodiments, analyzing the amniotic fluid fetal DNA to obtain a fragment size distribution pattern comprises: submitting the amniotic fluid fetal DNA to one or more of: gel electrophoresis, capillary gel electrophoresis, flow cytometry and MALDI-TOF mass spectrometry analysis. In certain preferred embodiments, the amniotic fluid fetal DNA is submitted to a gel electrophoresis analysis.

In certain embodiments, providing a prenatal diagnosis comprises one or more of: detecting a chromosomal abnormality, identifying a chromosomal abnormality, and identifying a disease or condition associated with a chromosomal abnormality affecting the fetus.

The methods of the invention may be performed for a fetus suspected of having a disease or condition associated with a chromosomal abnormality, for example an aneuploidy, such as Down syndrome, Patau syndrome, Edward syndrome, Turner syndrome, Klinefelter syndrome, and XYY disease. Alternatively or additionally, the methods of the invention may be performed for a fetus carried by a woman who is 35 or more than 35 years old.

In certain embodiments, the methods of the invention further comprise: comparing the fragment size distribution pattern obtained to at least one fragment size distribution pattern obtained for a control sample of amniotic fluid fetal DNA, prior to providing a prenatal diagnosis. The control sample of amniotic fluid fetal DNA may be from a karyotypically and developmentally normal fetus, or from a fetus with an identified chromosomal abnormality.

In other embodiments, the methods of the invention further comprise: repeating all the steps of the method for a statistically significant number of amniotic fluid fetal DNA samples from karyotypically and developmentally normal fetuses; and using the fragment size distribution patterns obtained to establish a fragment size distribution map for amniotic fluid fetal DNA from karyotypically and developmentally normal fetuses.

In still other embodiments, the methods of the invention further comprise: repeating all the steps of the method for a statistically significant number of amniotic fluid fetal DNA samples from fetuses with an identical chromosomal abnormality; and using the fragments size distribution patterns obtained to establish a fragment size distribution map for amniotic fluid fetal DNA from fetuses with that particular chromosomal abnormality.

In yet other embodiments, the methods of the invention further comprise: comparing the fragment size distribution pattern obtained to at least one fragment size distribution map prior to providing a prenatal diagnosis. The fragment size distribution map may be characteristic of a normal karyotype or characteristic of a particular chromosomal abnormality.

In another aspect, the present invention provides kits for prenatal diagnosis. In certain embodiments, a kit of the invention comprises one or more of the following components: materials to extract fetal DNA from a sample of amniotic fluid; materials to analyze amniotic fluid fetal DNA to obtain a fragment size distribution pattern; at least one fragment size distribution map; and instructions for using the kit for providing prenatal diagnosis according to the present invention.

These and other objects, advantages and features of the present invention will become apparent to those of ordinary skill in the art having read the following detailed description of the preferred embodiments.

### BRIEF DESCRIPTION OF THE DRAWING

FIG. 1 is a graph showing a comparison of the yield of cell-free fetal DNA (GAPDH locus) extracted from amniotic fluid supernatant from euploid singleton pregnancies. “0” indicates use of the new extraction protocol (as described in Example 2) and “1” use of the original extraction protocol (as described in Example 1 and in P. B. Larrabee et al., Am. J. Hum. Genet., 2004, 75: 485-491). The lines inside the boxes denote medians. The box indicates 25^{th} and 75^{th} percentiles. The whiskers denote the 10^{th} and 90^{th} percentiles. Symbols indicate data points outside the 10^{th} and 90^{th} percentiles.

FIG. 2 is a set of three graphs showing the correlation between GAPDH concentration and gestational age for (A) euploid fetuses, (B) fetuses with trisomy 21, and (C) fetuses with trisomy 18. In these experiments, cell-free fetal DNA was extracted following the improved extraction protocol and the quantity of total DNA was determined using real-time PCR (Applied Biosystems) using GAPDH locus (as described in Example 2). The results were obtained using 10 mL of frozen amniotic fluid supernatant from (A) 32 euploid fetuses (median gestational age [GA]: 16.9 weeks), Correlation coefficient: 0.78 (p<0.0001), R²: 0.396; (B) 17 fetuses with trisomy 21 (median GA: 16.4 weeks), Correlation coefficient: 0.11 (p=0.66), R²: 5.3×10⁻⁴; and (C) 7 fetuses with trisomy 18 (median GA: 16.5 weeks), Correlation coefficient: 0.36 (p=0.38), R²: 0.152.

FIG. 3 is a set of four graphs showing the fragmentation signature from cell-free fetal DNA samples from (A) euploid fetuses, (B) trisomy 21 fetuses, (C) trisomy 18 fetuses, and (D) trisomy 13 fetuses. In these experiments, cell-free fetal DNA was extracted following the improved extraction protocol (as described in Example 2) and gel electrophoresis (1% agarose) was performed to determine the fragmentation pattern of each sample using GeneTool (Syngene). In each of these graphs, the X axis represents run distance on the gel, expressed as R_{f} (retention factor), which is the distance migrated by a band divided by the distance migrated by the dye front. The Y axis represents fluorescence intensity of the electrophoretic profile. Each line represents a separate sample.

Table 1. Demographic variables and cell-free fetal DNA amplification results.

Table 2. Statistical analyses of cell-free DNA fragmentation signature [Median (25^{th}, 75^{th} percentiles).

### DEFINITIONS

Unless otherwise stated, all technical and scientific terms used herein have the meaning commonly understood by a person skilled in the art to which this invention belongs. The following terms have the meaning ascribed to them unless specified otherwise.

As used herein, the term “prenatal diagnosis” refers to the determination of the health and conditions of a fetus, including the detection of defects or abnormalities as well as the diagnosis of diseases. A variety of non-invasive and invasive techniques are available for prenatal diagnosis. Each of them can be used only during specific time periods of the pregnancy for greatest utility. These techniques include, for example, ultrasonography, maternal serum screening, amniocentesis, and chorionic villus sampling (or CVS). The methods of prenatal diagnosis of the present invention include the analysis of the fragment size distribution pattern of cell-free fetal DNA isolated from amniotic fluid. The inventive methods of prenatal diagnosis allow for determination of fetal characteristics such as chromosomal abnormality, and for identification of diseases or conditions associated with chromosomal abnormalities.

The terms “sonographic examination”, “ultrasonographic examination”, and “ultrasound examination” are used herein interchangeably. They refer to a clinical non-invasive procedure in which high frequency sound waves are used to produce visible images from the pattern of echos made by different tissues and organs of the fetus. A sonographic examination may be used to determine the size and position of the fetus, the size and position of the placenta, the amount of amniotic fluid, and the appearance of fetal anatomy. Ultrasound examinations can reveal the presence of congenital anomalies (i.e., anatomical or structural malformations that are present at birth).

The term “amniocentesis”, as used herein, refers to a prenatal test performed by inserting a long needle in the mother's lower abdomen into the amniotic cavity inside the uterus using ultrasound to guide the needle, and withdrawing a small amount of amniotic fluid. The amniotic fluid contains skin, kidney, and lung cells from the fetus. In conventional amniocentesis, these cells are grown in culture and tested for chromosomal abnormalities by determination and analysis of their karyotypes and the amniotic fluid itself can be tested for biochemical abnormalities. As discovered by the Applicants (see below), the amniotic fluid also contains cell-free fetal DNA.

The term “chromosome” has herein its art understood meaning. It refers to structures composed of very long DNA molecules (and associated proteins) that carry most of the hereditary information of an organism. Chromosomes are divided into functional units called “genes”, each of which contains the genetic code (i.e., instructions) for making a specific protein or RNA molecule. In humans, a normal body cell contains 46 chromosomes; a normal reproductive cell contains 23 chromosomes.

The terms “chromosomal abnormality”, “chromosomal aberration” and “chromosomal alteration” are used herein interchangeably. They refer to a difference (i.e., a variation) in the number of chromosomes or to a difference (i.e., a modification) in the structural organization of one or more chromosomes as compared to chromosomal number and structural organization in a karyotypically normal individual. As used herein, these terms are also meant to encompass abnormalities taking place at the gene level. The presence of an abnormal number of (i.e., either too many or too few) chromosomes is called “aneuploidy”. Examples of aneuploidy include trisomy 21, trisomy 18 and trisomy 13. Structural chromosomal abnormalities include: deletions (e.g., absence of one or more nucleotides normally present in a gene sequence, absence of an entire gene, or missing portion of a chromosome), additions (e.g., presence of one or more nucleotides usually absent in a gene sequence, presence of extra copies of a gene (also called duplication), or presence of an extra portion of a chromosome), rings, breaks and chromosomal rearrangements. Abnormalities that involve deletions or additions of chromosomal material alter the gene balance of an organism and if they disrupt or delete active genes, they generally lead to fetal death or to serious mental and physical defects. Structural rearrangements of chromosomes result from chromosome breakage caused by damage to DNA, errors in recombination, or crossing over the maternal and paternal ends of the separated double helix during meiosis or gamete cell division. Chromosomal rearrangements may be translocations or inversions. A translocation results from a process in which genetic material is transferred from one gene to another. A translocation is balanced when two chromosomes exchange pieces without loss of genetic material, while an unbalanced translocation occurs when chromosomes either gain or lose genetic material. Translocations may involve two chromosomes or only one chromosome. Inversions are produced by a process in which two breaks occur in a chromosome and the broken segment rotates 180°, resulting in the genes being rearranged in reverse order.

As used herein, the term “disease or condition associated with a chromosomal abnormality” refers to any disease, disorder, condition or defect, that is known or suspected to be caused by a chromosomal abnormality. Exemplary diseases or conditions associated with a chromosomal abnormality include, but are not limited to, trisomies (e.g., Down syndrome, Edward syndrome, Patau syndrome, Turner syndrome, Klinefelter syndrome, and XYY disease), and X-linked disorders (e.g., Duchenne muscular dystrophy, hemophilia A, certain forms of severe combined immunodeficiency, Lesch-Nyhan syndrome, and Fragile X syndrome). Additional examples of diseases or conditions associated with chromosomal abnormalities are given below and may also be found in “Harrison's Principles of Internal Medicine”, Wilson et al. (Ed.), 1991 (12^{th} Ed.), Mc Graw Hill: New York, N.Y., pp 24-46, which is incorporated herein by reference in its entirety.

As used herein, the term “karyotype” refers to the particular chromosome complement of an individual or a related group of individuals, as defined by the number and morphology of the chromosomes usually in mitotic metaphase. More specifically, a karyotype includes such information as total chromosome number, copy number of individual chromosome types (e.g., the number of copies of chromosome Y) and chromosomal morphology (e.g., length, centromeric index, connectedness and the like). Examination of a karyotype allows detection and identification of chromosomal abnormalities (e.g., extra, missing, or broken chromosomes). Since certain diseases and conditions are associated with characteristic chromosomal abnormalities, analysis of a karyotype allows diagnosis of these diseases and conditions.

The term “karyotypically and developmentally normal fetus” is used herein to designate a fetus whose karyotype is normal (i.e., it does not contain chromosomal abnormalities) and whose development has been determined to be appropriate for gestational age, for example, by sonographic examination.

As used herein, the term “statistically significant number” refers to a number of samples (analyzed or to be analyzed) that is large enough to provide reliable data.

As used herein, the term “G (or Giemsa) banding” refers to a standard staining technique for karyotyping. G-banding (also known as G-T-G banding) involves the use of an enzyme (the protease trypsin) to degrade some of the proteins that are associated with the chromosomes and the use of a staining dye (Giemsa) that selectively binds to DNA regions rich in guanine and cytosine. This selective staining leads to the formation of a distinctive pattern of alternating dark and light bands along the length of the chromosome, that is characteristic of the individual chromosome (light bands correspond to euchromatin, which is active DNA rich in guanine and cytosine; dark bands correspond to heterochromatin, which is unexpressed DNA rich in adenine and thymine). This staining reveals extra and missing chromosomes, large deletions and duplications, as well as the locations of centromeres (the major constrictions in chromosomes). However less extensive or more complex rearrangements of genetic material, chromosomal origins of markers, and subtle translocations are not detectable or are difficult to identify with certainty using standard G-banding (Giemsa, Leishman's or variant). For more details on how to perform a G-banding analysis, see, for example, J. M. Scheres et al., Hum. Genet. 1982, 61: 8-11; and K. Wakui et al., J. Hum. Genet. 1999, 44: 85-90, each of which if incorporated herein by reference in its entirety.

As used herein, the term “Fluorescence In Situ Hybridization or FISH” refers to a molecular cytogenetic technique that can be used to generate karyotypes. In a FISH experiment, specifically designed fluorescent molecules are used to visualize particular genes or sections of chromosomes by fluorescence microscopy, thus allowing detection of chromosomal abnormalities. FISH on interphase nuclei (mainly from uncultured amniocytes) is an increasingly popular tool for the rapid exclusion of selected aneuploidies (see, for example, T. Bryndorf et al., Acta Obstet. Gynecol. Scand, 2000, 79: 8-14; W. Cheong Leung et al., Prenat. Diagn. 2001, 21: 327-332; J. Pepperberg et al., Prenat. Diagn. 2001, 21: 293-301; S. Weremowicz et al., Prenat. Diagn. 2001, 21: 262-269; and R. Sawa et al., J. Obstet. Gynaecol. Res. 2001, 27: 41-47, each of which is incorporated herein by reference in its entirety).

As used herein, the term “Spectral Karyotyping or SKY”, refers to a molecular cytogenetic technique that allows for the simultaneous visualization of all human (or mouse) chromosomes in different colors, which considerably facilitates karyotype analysis. SKY involves the preparation of a library of short sequences of single-stranded DNA labeled with spectrally distinguishable fluorescent dyes. Each of the individual probes in this DNA library is complementary to a unique region of a chromosome, while together all the probes make up a collection of DNA that is complementary to all of the chromosomes within the human genome. After in situ hybridization, the measurement of defined emission spectra by spectral imaging allows for the definitive discernment of all human chromosomes in different colors and the detection of chromosomal abnormalities, such as translocations, chromosomal breakpoints, and rearrangements. For more details about the SKY technique and its use in determining karyotypes, see, for example, E. Shrock et al., Hum. Genet. 1997, 101: 255-262; I. B. Van den Veyver and B. B. Roa, Curr. Opin. Obstet. Gynecol. 1998, 10: 97-103; M. C. Phelan et al., Prenatal Diagn. 1998, 18: 1174-1180; B. R. Haddad et al., Hum. Genet. 1998, 103: 619-625; and B. Peschka et al., Prenatal. Diagn. 1999, 19: 1143-1149, each of which is incorporated herein by reference in its entirety.

The terms “comparative genomic hybridization or CGH” and “metaphase comparative genomic hybridization or metaphase CGH” are used herein interchangeably. They refer to a molecular cytogenetic technique that involves differentially labeling a test DNA and normal reference DNA with fluorescent dyes, co-hybridizing the two labeled DNA samples to normal metaphase chromosome spreads, and visualizing the two hybridized DNAs by fluorescence. The ratio of the intensity of the two fluorescent dyes along a certain chromosome or chromosomal region reflects the relative copy number (i.e., abundance) of the respective nucleic acid sequences in the two samples. A CGH analysis provides a global overview of gains and losses of genetic material throughout the whole genome. As used herein, the term “standard metaphase chromosome analysis” refers to conventional G-banding analysis or metaphase CGH.

The term “nucleic acid” and “nucleic acid molecule” are used herein interchangeably. They refer to a deoxyribonucleotide or ribonucleotide polymer in either single-or double-stranded form, and unless otherwise stated, encompass known analogs of natural nucleotides that can function in a similar manner as naturally occurring nucleotides. The terms encompass nucleic acid-like structures with synthetic backbones, as well as amplification products.

The terms “genomic DNA” and “genomic nucleic acid” are used herein interchangeably. They refer to nucleic acid isolated from a nucleus of one or more cells, and include nucleic acid derived from (i.e., isolated from, amplified from, cloned from as well as synthetic versions of) genomic DNA. Fetal DNA isolated from amniotic fluid may be considered as genomic DNA as it was found to represent the entire genome equally.

The term “sample of DNA” (as used for example, in “sample of amniotic fluid fetal DNA”) refers to a sample comprising DNA or nucleic acid representative of DNA isolated from a natural source and in a form suitable for analysis (e.g., as a soluble aqueous solution). Samples of DNA to be used in the practice of the present invention include a plurality of nucleic acid segments (or fragments) which together cover a substantially complete genome.

As used herein, a “plurality” of elements refers to 2 or more elements.

The terms “DNA fragment” and “nucleic acid fragment” are used herein interchangeably and refer to a polynucleotide sequence obtained from a genome at any point along the genome and encompassing any sequence of nucleotides.

The terms “fragment size pattern”, “fragment size distribution pattern”, and “fragmentation pattern” are used herein interchangeably. A fragment size pattern may include information regarding one or more of: the total number of nucleic acid fragments present in a sample, the size of one or more nucleic acid fragments in the sample, the absolute or relative abundance levels of nucleic acid fragments of a specific size or size range, and the absolute or relative abundance levels of nucleic acid fragments of different size present in the sample.

The term “fragment size”, as used herein in reference to a nucleic acid molecule, refers to the number of base pairs of the nucleic acid, which denotes the length of the molecule.

The term “hybridization” refers to the binding of two single stranded nucleic acids via complementary base pairing. Hybridization between two nucleic acid molecules includes minor mismatches that can be accommodated by reducing the stringency of the hybridization/wash media to achieve the desired detection of the sequence of interest.

In the context of the present invention, the term “fetal genomic information” refers to any kind of information that can be extracted through analysis of amniotic fluid fetal DNA. Fetal genomic information includes, for example, gain and loss of genetic material, chromosomal abnormalities and genome copy number changes or ratios at multiple genomic loci.

The term “made available for analysis” is used herein to specify that amniotic fluid fetal DNA is manipulated (e.g., amplified, labeled, cloned, purified, and/or concentrated and resuspended in a soluble aqueous solution) such that it is in a form suitable for analysis (e.g., by gel electrophoresis).

The term “Polymerase Chain Reaction or PCR” has herein its art understood and refers to a technique for making multiple copies of a specific stretch of DNA or RNA. PCR can be used to test for mutations in DNA. PCR can also be used to quantify the amount of nucleic acid in a sample, to sub-clone, or to label nucleic acid molecules. Methods of performing PCR experiments are well known in the art.

The terms “labeled”, “labeled with a detectable agent”, and “labeled with a detectable moiety” are used herein interchangeably. They are used herein to specify that a nucleic acid molecule or individual nucleic acid segments from a sample can be visualized. Preferably, the detectable agent or moiety is selected such that it generates a signal which can be measured and whose intensity is related to the amount of nucleic acids. Methods for labeling nucleic acid molecules are well known in the art (see below for a more detailed description of such methods). Labeled nucleic acid fragments can be prepared by incorporation of or conjugation to a label, that is directly or indirectly detectable by spectroscopic, photochemical, biochemical, immunochemical, electrical, optical, or chemical means. Suitable detectable agents include, but are not limited to: various ligands, radionuclides, fluorescent dyes, chemiluminescent agents, microparticles, enzymes, calorimetric labels, magnetic labels, and haptens. Detectable moieties can also be biological molecules such as molecular beacons and aptamer beacons.

The terms “fluorophore”, “fluorescent moiety”, “fluorescent label”, “fluorescent dye” and “fluorescent labeling moiety” are used herein interchangeably. They refer to a molecule which, in solution and upon excitation with light of appropriate wavelength, emits light back. Numerous fluorescent dyes of a wide variety of structures and characteristics are suitable for use in the practice of this invention. Similarly, methods and materials are known for fluorescently labeling nucleic acids (see, for example, R. P. Haugland, “Molecular Probes: Handbook of Fluorescent Probes and Research Chemicals 1992-1994”, 5^{th} Ed., 1994, Molecular Probes, Inc., which is incorporated herein by reference in its entirety). In choosing a fluorophore, it is preferred that the fluorescent molecule absorbs light and emits fluorescence with high efficiency (i.e., it has a high molar absorption coefficient and a high fluorescence quantum yield, respectively) and is photostable (i.e., it does not undergo significant degradation upon light excitation within the time necessary to perform the array-based hybridization analysis.

### DETAILED DESCRIPTION OF CERTAIN PREFERRED EMBODIMENTS

The present invention is directed to improved strategies for prenatal diagnosis, screening, monitoring and/or testing. In particular, systems are described that allow for the rapid assessment of fetal characteristics such as chromosomal abnormalities and for the prenatal diagnosis of a variety of diseases and conditions.

The Applicants have previously shown that amniotic fluid is a rich source of fetal nucleic acids, and that analysis of cell-free fetal DNA isolated from amniotic fluid by array-based hybridization techniques such as genomic microarrays, provides a “molecular karyotype” of the fetus, which contains more complete and/or more detailed information than is obtained using a standard banding method (U.S. application Ser. No. 10/577,341 and PCT application No. PCT/US2004/035929, both entitled “Prenatal Diagnosis using Cell-Free Fetal DNA in Amniotic Fluid”, each of which is incorporated herein by reference in its entirety).

The present invention encompasses the recognition, by the Applicants, that cell-free fetal DNA isolated from amniotic fluid has a fragment size pattern that is different in karyotypically normal fetuses and in fetuses with a chromosomal abnormality. Furthermore, the fragment size pattern was found to be characteristic for each type of chromosomal abnormality, acting as a “fingerprint” or “signature” of the presence of the chromosomal abnormality in a fetus' karyotype. Accordingly, the present invention provides novel approaches for the rapid detection of chromosomal abnormalities in fetuses and for the prenatal diagnosis of diseases and conditions associated with chromosomal abnormalities using fragment size pattern of cell-free fetal DNA from amniotic fluid.

I. Cell-Free Fetal DNA from Amniotic Fluid

The methods of the invention involve analysis of the fragmentation pattern of cell-free fetal DNA isolated from amniotic fluid. Work carried out in the Applicants' laboratory (D. W. Bianchi et al., Clin. Chem. 2001, 47: 1867-1869, which is incorporated herein by reference in its entirety) has demonstrated that cell-free fetal DNA is present in large amounts in the amniotic fluid and that it can be isolated easily using standard procedures.

Amniotic Fluid Sample

Practicing the methods of the invention involves providing a sample of amniotic fluid obtained from a pregnant woman. Amniotic fluid is generally collected using a method called amniocentesis, in which a long needle is inserted in the mother's lower abdomen into the amniotic cavity inside the uterus; and a small amount of amniotic fluid is withdrawn.

For prenatal diagnosis, most amniocenteses are performed between the 14^{th} and 20^{th} weeks of pregnancy. The most common indications for amniocentesis include: advanced maternal age (typically set, in the US, at 35 or more than 35 years at the estimated time of delivery), previous child with a birth defect or genetic disorder, parental chromosomal rearrangement, family history of late-onset disorders with genetic components, recurrent miscarriages, positive maternal serum screening test (Multiple Marker Screening) documenting increased risk of fetal neural tube defects and/or fetal chromosomal abnormality, and abnormal fetal ultrasound examination (for example, revealing signs known to be associated with fetal aneuploidy). Risks with amniocentesis are uncommon, but include fetal loss and maternal Rh sensitization. The increased risk of fetal mortality following amniocentesis is about 0.5 to 1% above what would normally be expected. Side effects to the mother include cramping, bleeding, infection and leaking of amniotic fluid following the procedure.

Amniocentesis is presently one of the clinical tests that detect the greatest variety of fetal impairments. In conventional amniocentesis procedures, fetal cells present in the amniotic fluid are isolated by centrifugation and grown in culture for chromosome analysis, biochemical analysis and molecular biological analysis. Centrifugation, which removes cell populations from the amniotic fluid, also produces a supernatant sample (herein termed “remaining amniotic material”). This sample is usually stored at −20° C. as a back-up in case of assay failure. Aliquots of this supernatant may also be used for additional assays such as determination of alpha-fetoprotein and acetyl cholinesterase levels. After a certain period of time, the frozen supernatant sample is typically discarded. The standard protocol followed by the Cytogenetics Laboratory at Tufts-New England Medical Center (Boston, Mass.), which provides samples of remaining amniotic material to the Applicants is described in detail in Example 1.

Isolation of Cell-Free Fetal DNA

Cell-free fetal DNA for use in the methods of the present invention is isolated from a sample of amniotic fluid obtained from a pregnant woman. The isolation may be carried out by any suitable method of DNA isolation or extraction.

In certain preferred embodiments, cell-free fetal DNA is isolated from the remaining amniotic material obtained after removal of cell populations from a sample of amniotic fluid. The cell populations may be removed from the amniotic fluid by any suitable method, for example, by centrifugation.

In certain embodiments, substantially all the cell populations are removed from the amniotic fluid, for example, by performing more than one centrifugation. In other embodiments, the remaining amniotic material (i.e., the material obtained after cell removal) includes some cell populations.

As already mentioned above, before isolation or extraction of cell-free fetal DNA, the remaining amniotic material may be frozen and stored for a certain period of time under suitable storage conditions. Fetal DNA stored at −20° C. for up to 8 years was found to be suitable for analysis. Before extraction, the frozen sample may be thawed at 37° C. and then mixed with a vortex. Any remaining cell populations still present in the amniotic fluid sample may be eliminated by centrifugation.

Isolating fetal DNA includes treating the remaining amniotic material such that cell-free fetal DNA present in the remaining amniotic material is extracted and made available for analysis. Any suitable isolation method that results in extracted amniotic fluid fetal DNA may be used in the practice of the invention.

Methods of DNA extraction are well known in the art. A classical DNA isolation protocol is based on extraction using organic solvents such as a mixture of phenol and chloroform, followed by precipitation with ethanol (see, for example, J. Sambrook et al., “Molecular Cloning: A Laboratory Manual”, 1989, 2^{nd} Ed., Cold Spring Harbour Laboratory Press: New York, N.Y.). Other methods include: salting out DNA extraction (see, for example, P. Sunnucks et al., Genetics, 1996, 144: 747-756; and S. M. Aljanabi and I. Martinez, Nucl. Acids Res. 1997, 25: 4692-4693); the trimethylammonium bromide salts DNA extraction method (see, for example, S. Gustincich et al., BioTechniques, 1991, 11: 298-302) and the guanidinium thiocyanate DNA extraction method (see, for example, J. B .W. Hammond et al., Biochemistry, 1996, 240: 298-300).

There are also numerous different and versatile kits that can be used to extract DNA from bodily fluids and that are commercially available from, for example, BD Biosciences Clontech (Palo Alto, Calif.), Epicentre Technologies (Madison, Wis.), Gentra Systems, Inc. (Minneapolis, Minn.), MicroProbe Corp. (Bothell, Wash.), Organon Teknika (Durham, N.C.), and Qiagen Inc. (Valencia, Calif.). User Guides that describe in great detail the protocol to be followed are usually included in all these kits. Sensitivity, processing time and cost may be different from one kit to another. One of ordinary skill in the art can easily select the kit(s) most appropriate for a particular situation.

Typically, fetal DNA extraction is carried out on aliquots of from about 8 mL to about 15 mL of remaining amniotic material. Preferably, the extraction is carried out on an aliquot of from about 12 mL to about 15 mL of remaining amniotic material. However, the extraction may be carried out on an aliquot of more than 15 mL of remaining amniotic material.

When substantially all cell populations are removed from the sample of amniotic fluid, the amniotic fluid fetal DNA consists essentially of cell-free fetal DNA. When only part of all the cell populations are removed from the sample of amniotic fluid, the amniotic fetal DNA comprises cell-free fetal DNA as well as DNA originating from the cells that were still present in the remaining amniotic material. In the latter case, a larger amount of DNA is generally obtained.

DNA extractions carried out, by the Applicants, on samples of remaining amniotic material of ≧10 mL in volume, using the “Blood and Body Fluid” protocol as described by Qiagen, yielded between 8 and 900 ng of fetal DNA. Cell-free fetal DNA isolated from amniotic fluid was found to represent the whole genome equally (P. B. Larrabee et al., Am. J. Hum. Genet., 2004, 75: 485-491, which is incorporated herein by reference in its entirety).

Improved Method for the Isolation of Cell-Free Fetal DNA from Amniotic Fluid

Preferably, cell-free fetal DNA is extracted from amniotic fluid using an improved extraction protocol developed by the Applicants (see U.S. Provisional Application No. 60/714,035, which is incorporated herein by reference in its entirety). Compared to the “Blood and Body Fluid” vacuum protocol (Qiagen, Valencia, Calif.), this improved method of extraction is more rapid and leads to increased recovery yields of high quality fetal DNA.

The extraction method originally used (see Example 1) was based on known protocols for the isolation of cell-free fetal DNA from maternal plasma/serum, as specific guidelines for the extraction of DNA from amniotic fluid did not exist. Optimization of the isolation protocol by the Applicants, led to the first method specifically adapted to the extraction of cell-free fetal DNA from amniotic fluid supernatant.

More specifically, increased yields of extracted cell-free fetal DNA were obtained from 10 mL samples of amniotic fluid when the original method using the “Blood and Body Fluid” vacuum protocol was modified as follows: (1) the vacuum extraction pressure was increased to 800 mbar, (2) Maxi Spin Columns were used instead of Mini Spin Columns, and (3) the AL buffer was replaced with AVL buffer supplemented with DNA carrier for the extraction of low concentrations of target DNA. The replacement of AL buffer with AVL buffer eliminates the need for the heating bath during the lysis step.

As reported in Example 2, these modifications in the extraction protocol leads to a high increase in the yield of fetal DNA extracted from amniotic fluid supernatant and to a significantly larger proportion of samples containing more than 100 ng of extracted DNA. Furthermore, the modified protocol involves fewer steps, which lowers the chance of potential contamination and also speeds up the isolation process allowing for the extraction of cell-free fetal DNA from up to 10 (10 mL) amniotic fluid supernatant samples in less than 3 hours.

Amplification of Extracted Amniotic Fluid Fetal DNA

In certain embodiments, amplification is used to quantify the amount of extracted fetal DNA (see, for example, U.S. Pat. No. 6,294,338).

Amplification methods are well known in the art (see, for example, A. R. Kimmel and S. L. Berger, Methods Enzymol. 1987, 152: 307-316; J. Sambrook et al., “Molecular Cloning: A Laboratory Manual”, 1989, 2^{nd} Ed., Cold Spring Harbour Laboratory Press: New York, N.Y.; “Short Protocols in Molecular Biology”, F. M. Ausubel (Ed.), 2002, 5^{th} Ed., John Wiley & Sons; U.S. Pat. Nos. 4,683,195; 4,683,202 and 4,800,159). Standard nucleic acid amplification methods include: polymerase chain reaction (or PCR, see, for example, “PCR Protocols: A Guide to Methods and Applications”, M. A. Innis (Ed.), Academic Press: New York, 1990; and “PCR Strategies”, M. A. Innis (Ed.), Academic Press: New York, 1995); ligase chain reaction (or LCR, see, for example, U. Landegren et al., Science, 1988, 241: 1077-1080; and D. L. Barringer et al., Gene, 1990, 89: 117-122); transcription amplification (see, for example, D. Y. Kwoh et al., Proc. Natl. Acad. Sci. USA, 1989, 86: 1173-1177); self-sustained sequence replication (see, for example, J. C. Guatelli et al., Proc. Natl. Acad. Sci. USA, 1990, 87: 1874-1848); Q-beta replicase amplification (see, for example, J. H. Smith et al., J. Clin. Microbiol. 1997, 35: 1477-1491); automated Q-beta replicase amplification assay (see, for example, J. L. Burg et al., Mol. Cell. Probes, 1996, 10: 257-271) and other RNA polymerase mediated techniques such as, for example, nucleic acid sequence based amplification (or NASBA, see, for example, A. E. Greijer et al., J. Virol. Methods, 2001, 96: 133-147).

A PCR method for the quantification of fetal DNA extracted from amniotic fluid is described in Example 2.

Alternatively, other quantification methods may be used including, but not limited to, digestion with restriction endonuclease, ultraviolet light visualization of ethidium bromide stained agarose gels; DNA sequencing, or hybridization with allele specific oligonucleotide probes (R. K. Saiki et al., Am. J. Hum. Genet. 1988, 43(suppl.): A35).

Labeling of Amniotic Fluid Fetal DNA

In certain embodiments, extracted fetal DNA is labeled with a detectable agent or moiety before being analyzed. The role of a detectable agent is to allow visualization of nucleic acid fragments under analysis conditions. Preferably, the detectable agent is selected such that it generates a signal which can be measured and whose intensity is related (e.g., proportional) to the amount of labeled nucleic acids present in the sample being analyzed.

The association between the nucleic acid molecule and detectable agent can be covalent or non-covalent. Labeled nucleic acid fragments can be prepared by incorporation of or conjugation to a detectable moiety. Labels can be attached directly to the nucleic acid fragment or indirectly through a linker. Linkers or spacer arms of various lengths are known in the art and are commercially available, and can be selected such that they reduce steric hindrance, and/or confer other useful or desired properties to the resulting labeled molecules (see, for example, E. S. Mansfield et al., Mol. Cell. Probes, 1995, 9: 145-156).

Methods for labeling nucleic acid fragments are well-known in the art. For a review of labeling protocols, label detection techniques and recent developments in the field, see, for example, L. J. Kricka, Ann. Clin. Biochem. 2002, 39: 114-129; R. P. van Gijlswijk et al., Expert Rev. Mol. Diagn. 2001, 1: 81-91; and S. Joos et al., J. Biotechnol. 1994, 35: 135-153. Standard nucleic acid labeling methods include: incorporation of radioactive agents, direct attachment of fluorescent dyes (see, for example, L. M. Smith et al., Nucl. Acids Res. 1985, 13: 2399-2412) or of enzymes (see, for example, B. A. Connoly and P. Rider, Nucl. Acids. Res. 1985, 13: 4485-4502); chemical modifications of nucleic acid fragments making them detectable immunochemically or by other affinity reactions (see, for example, T. R. Broker et al., Nucl. Acids Res. 1978, 5: 363-384; E. A. Bayer et al., Methods of Biochem. Analysis, 1980, 26: 1-45; R. Langer et al., Proc. Natl. Acad. Sci. USA, 1981, 78: 6633-6637; R. W. Richardson et al., Nucl. Acids Res. 1983, 11: 6167-6184; D. J. Brigati et al., Virol. 1983, 126: 32-50; P. Tchen et al., Proc. Natl Acad. Sci. USA, 1984, 81: 3466-3470; J. E. Landegent et al., Exp. Cell Res. 1984, 15: 61-72; and A. H. Hopman et al., Exp. Cell Res. 1987, 169: 357-368); and enzyme-mediated labeling methods, such as random priming, nick translation, PCR and tailing with terminal transferase (for a review on enzymatic labeling, see, for example, J. Temsamani and S. Agrawal, Mol. Biotechnol. 1996, 5: 223-232). More recently developed nucleic acid labeling systems include, but are not limited to: ULS (Universal Linkage System), which is based on the reaction of monoreactive cisplatin derivatives with the N7 position of guanine moieties in DNA (see, for example, R. J. Heetebrij et al., Cytogenet. Cell. Genet. 1999, 87: 47-52), psoralen-biotin, which intercalates into nucleic acids and becomes covalently bonded to the nucleotide bases upon UV irradiation (see, for example, C. Levenson et al., Methods Enzymol. 1990, 184: 577-583; and C. Pfannschmidt et al., Nucleic Acids Res. 1996, 24: 1702-1709), photoreactive azido derivatives (see, for example, C. Neves et al., Bioconjugate Chem. 2000, 11: 51-55), and DNA alkylating agents (see, for example, M. G. Sebestyen et al., Nat. Biotechnol. 1998, 16: 568-576).

Any of a wide variety of detectable agents can be used in the practice of the present invention. Suitable detectable agents include, but are not limited to: various ligands, radionuclides (such as, for example, ³²p, ³⁵s, ³H, ¹⁴C, ¹²⁵I, ¹³¹I, and the like); fluorescent dyes (for specific exemplary fluorescent dyes, see below); chemiluminescent agents (such as, for example, acridinium esters, stabilized dioxetanes and the like); microparticles (such as, for example, quantum dots, nanocrystals, phosphors and the like); enzymes (such as, for example, those used in an ELISA, i.e., horseradish peroxidase, beta-galactosidase, luciferase, alkaline phosphatase); colorimetric labels (such as, for example, dyes, colloidal gold and the like); magnetic labels (such as, for example, Dynabeads™); and biotin, dioxigenin or other haptens and proteins for which antisera or monoclonal antibodies are available.

In certain embodiments, amniotic fluid fetal DNA to be analyzed is fluorescently labeled. Numerous known fluorescent labeling moieties of a wide variety of chemical structures and physical characteristics are suitable for use in the practice of this invention. Suitable fluorescent dyes include, but are not limited to: Cy-3™, Cy-5™, Texas red, FITC, Spectrum Red™, Spectrum Green™, phycoerythrin, rhodamine, fluorescein, fluorescein isothiocyanine, carbocyanine, merocyanine, styryl dye, oxonol dye, BODIPY dye (i.e., boron dipyrromethene difluoride fluorophore), and equivalents, analogues or derivatives of these molecules. Similarly, methods and materials are known for linking or incorporating fluorescent dyes to biomolecules such as nucleic acids (see, for example, R. P. Haugland, “Molecular Probes. Handbook of Fluorescent Probes and Research Chemicals 1992-1994”, 5^{th} Ed., 1994, Molecular Probes, Inc.). Fluorescent labeling agents as well as labeling kits are commercially available from, for example, Amersham Biosciences Inc. (Piscataway, N.J.), Molecular Probes Inc. (Eugene, Oreg.), and New England Biolabs Inc. (Berverly, Mass.).

Favorable properties of fluorescent labeling agents to be used in the practice of the invention include high molar absorption coefficient, high fluorescence quantum yield, and photostability. Preferred labeling fluorophores exhibit absorption and emission wavelengths in the visible (i.e., between 400 and 750 nm) rather than in the ultraviolet range of the spectrum (i.e., lower than 400 nm).

Detectable moieties can also be biological molecules such as molecular beacons and aptamer beacons. Molecular beacons are nucleic acid molecules carrying a fluorophore and a non-fluorescent quencher on their 5′ and 3′ ends. In the absence of a complementary nucleic acid strand, the molecular beacon adopts a stem-loop (or hairpin) conformation, in which the fluorophore and quencher are in close proximity to each other, causing the fluorescence of the fluorophore to be efficiently quenched by FRET (i.e., fluorescence resonance energy transfer). Binding of a complementary sequence to the molecular beacon results in the opening of the stem-loop structure, which increases the physical distance between the fluorophore and quencher thus reducing the FRET efficiency and resulting in emission of a fluorescence signal. The use of molecular beacons as detectable moieties is well-known in the art (see, for example, D. L. Sokol et al., Proc. Natl. Acad. Sci. USA, 1998, 95: 11538-11543; and U.S. Pat. Nos. 6,277,581 and 6,235,504). Aptamer beacons are similar to molecular beacons except that they can adopt two or more conformations (see, for example, O. K. Kaboev et al., Nucleic Acids Res. 2000, 28: E94; R. Yamamoto et al., Genes Cells, 2000, 5: 389-396; N. Hamaguchi et al., Anal. Biochem. 2001, 294: 126-131; S. K. Poddar and C. T. Le, Mol. Cell. Probes, 2001, 15: 161-167).

The selection of a particular nucleic acid labeling technique will depend on the situation and will be governed by several factors, such as the ease and cost of the labeling method, the quality of sample labeling desired, the nature and intensity of the signal generated by the detectable label, and the like.

II. Fragment Size Distribution Analysis of Amniotic Fluid Fetal DNA

As already mentioned above, the present invention provides methods of prenatal diagnosis, screening, monitoring, and/or testing, which include analysis of the fragment size distribution of cell-free fetal DNA isolated from amniotic fluid.

In the practice of the methods of the invention, DNA fragment size distribution analysis may be carried out by any method that can achieve size separation of components of a sample and provide information about the size and/or abundance of some or all of the different components of the sample. Examples of suitable methods include, but are not limited to, gel electrophoresis, capillary electrophoresis (CE) (R. A. Mathies and X. C. Huang, Nature, 1992, 359: 167-169), flow cytometry, and matrix-assisted laser desorption/ionization (MALDI) time-of-flight (TOF) spectrometry (K. J. Wu et al., Rapid Commun. Mass Spectrom., 1993, 7: 142-146).

Gel Electrophoresis

Gel electrophoresis involves moving a population of molecules (e.g., nucleic acid fragments) through an appropriate medium, such that the molecules are separated according to size. More specifically, an electric field is placed across a gel (in the form of a slab) containing the fragments causing the smaller fragments to move faster than the larger ones.

Gel electrophoresis is a well-known technique and has been used to produce band patterns of DNA fragments that form a fingerprint to identify the individual source of the DNA piece under analysis. The band patterns of specific DNA sequences are conventionally visualized by binding radioactive DNA probes to the separated DNA fragments and exposing suitable film to the radioactive labeled fragments (J. I. Thornton, “DNA Profiling”, C&EN, pp. 18-30, Nov. 20, 1989). In one variation, the fragment ends are tagged with a fluorescent dye so that the fragment migration time along a known path length in an electrophoretic gel can be determined by automated fluorescence detection (A. V. Carrano et al., Genomics, 1989, 4: 129-136).

Capillary Electrophoresis

Alternatively (or additionally), DNA fragment size analysis can be performed by capillary electrophoresis. Capillary electrophoresis (CE) has demonstrated its advantage over standard slab gel based electrophoresis techniques as a rapid, high-throughput and high-resolution method for separation of biological macromolecules, such as proteins, peptides and nucleic acids (G. W. Slater et al., Electrophoresis, 1998, 19: 1525-1541; A. Guttman and K. J. Ulfelder, Adv. Chromatogr., 1998, 38: 301-340). Capillary gel electrophoresis is the CE-analog of traditional slab-gel electrophoresis and is most often used for size-based separation of biological macromolecules such as oligonucleotides, DNA restriction fragments and proteins. The separation is performed by filling the capillary with a sieving matrix, for example, cross-linked polyacrylamide, agarose or linear polymer solutions. The main advantages over slab-gel electrophoresis are a wider range of gel matrixes and compositions, on-line detection, improved quantitation and automation.

More recent advances have allowed CE to be performed on arrays (X. C. Huang et al., Anal. Chem., 1992, 64: 2149-2154) and on microchip devices (J. Cheng et al., Anal. Biochem., 1998, 257: 101-106; S. C. Jacobson and J. M. Ramsey, Electrophoresis, 1995, 16: 481-486; L. C. Walters et al., Anal. Chem., 1998, 70: 158-162; J. Khandurina et al., Anal. Chem., 1999, 71: 1815-1819). The advent of photolithography has permitted micro-machining capillary electrophoresis channels in glass. Because of the small dimensions of the separation channels, separations may be performed even more rapidly than with conventional CE equipment (S. C. Jacobson and J. M. Ramsey, Anal. Chem., 1996, 68: 720-723).

New generations of CE instruments are commercially available, for example, from Agilent Technologies (Palo Alto, Calif.), CombiSep Inc. (Ames, Iowa), Molecular Dynamics (Sunnyvale, Calif.) and PE Applied Biosystems (Foster City, Calif.).

Flow Cytometry

DNA fragment sizing in the practice of the methods of the invention can, alternatively or additionally, be performed using methods based on flow cytometry (P. M. Goodwin et al., Nucl. Acids Res., 1993, 21: 803-806; Z. Huang et al., Nucl. Acids Res., 1996, 24: 4202-4209; X. Yan et al., Anal. Chem., 1999, 71: 5470-5480; each of which is incorporated herein by reference in its entirety)

Flow cytometry is a sensitive and quantitative technique that analyzes particles (such as stained/labeled nucleic acid fragments) in a fluid medium based on the particles' optical characteristics (for background information on flow cytometry, see, for example, H. M. Shapiro, “Practical Flow Cytometry”, 3^{rd} Ed., 1995, Alan R. Liss, Inc.; and “Flow Cytometry and Sorting, Second Edition”, Melamed et al. (Eds), 1990, Wiley-Liss: New York, which are incorporated herein by reference in their entirety). The fundamental concept of flow cytometry is simple. A flow cytometer hydrodynamically focuses a fluid suspension of particles which have been attached to one or more flurorophores, into a thin stream so that the particles flow down the stream in substantially single file and pass through an examination or analysis zone. A focused light beam, such as a laser beam, illuminates the particles as they flow through the examination zone. Optical detectors within the flow cytometer measure certain characteristics of the light as it interacts with the particles. Light interaction with the particles is generally measured as light scatter and particle fluorescence at one or more wavelengths.

MALDI-TOF

Alternatively or additionally, fetal DNA fragment sizing can be performed by MALDI-TOF mass spectrometry (J. A. Monforte and C. H. Becker, Nature Medicine, 1997, 3: 360-362; A. Stedding and C. H. Becker, Rapid Commun. Mass Spectrom., 1993, 7: 142-146). MALDI-TOF mass spectrometry provides for the spectrometric determination of the mass of poorly ionizing or easily-fragmented analytes of low volatility by embedding a matrix of light-absorbing material and measuring the weight of the molecule as it is ionized and caused to fly by volatilization. Combinations of electric and magnetic field are applied on the sample to cause the ionized material to move depending on the individual mass and charge of the molecule (see, for example, U.S. Pat. Nos. 5,288,644; 5,885,775; 5,905,259; 5,965,363; 6,002,127 and 6,043,031, each of which is incorporated herein by reference in its entirety).

Fragment Size Distribution Determination and Data Analysis

Any of a variety of methods and means may be used for determining the fragment size distribution of fetal DNA after fragment separation according to size. Except for mass spectrometry methods, which directly provide the mass of each separate fragment, fragment size determination generally involves detection of the fragment labels, which generate a signal that permits characterization of the size and quantity of the DNA fragments.

The labels can be radioactive, fluorescence, infrared, or other non-radioactive labels (“Current Protocols in Molecular Biology”, F. M. Ausubel et al. (Eds), 1995, John Wiley and Sons, New York; N.Y.; “Current Protocols in Human Genetics”, N. J. Dracopoli et al. (Eds.), 1995, John Wiley and Sons, New York; N.Y.; “Nonisotopic Probing, Blotting, and Sequencing”, L. J. Kricka et al. (Eds.), 1995, 2^{nd} Ed., Academic Press: San Diego, Calif.).

The label detection method will generally depend on both the label(s) used and the size separation mechanism. For example, radioactive labels can be detected using film or phosphor screens. Stained electrophoretic gels can be imaged using appropriate camera and films, and the images obtained can be scanned as described in Example 3. Scanners are also available for post-electrophoresis detection of DNA fragments. The DNA fragments may be fluorescently labeled with either intercalating dyes such as SYBR Green or end-labeled with different color dyes, such as FAM, JOE, HEX, etc. Using a scanner for fragment size distribution has the potential of high-throughput with digital data storage because multiple gels may be electrophoresed simultaneously off-line followed by a sequencing feeding to the scanner to record the band positions. With automated size separation methods (e.g., automated DNA sequencers, single capillary or capillary array instruments) the detection may be performed by laser scanning of the fluorescently labeled fragments, imaging on a CCD camera, and electronic acquisition of the signals from the CCD camera.

Size characterization may be done by comparing the sample fragment's signal in the context of the size standards. By separate calibration of the size standards used, the relative molecular size can be inferred. This size is usually only an approximation of the true size in base pair units, since the size standards and the sample fragments generally have different chemistries and electrophoretic migration patterns.

Quantification of the DNA signal is usually done by examining peak heights or peak areas taking into account band overlap between peaks. It is often useful to determine the quality (e.g., error, accuracy, concordance with expectations) of the size or quantity characterizations (D. R. Richards and M. W. Perlin, Am. J. Hum. Genet., 1995, 57: A26).

Softwares for the automatic analysis of gel images may be used for size characterization and quantification of the DNA signal. Such softwares are commercially available (e.g., GeneTools from Syngene (Frederick, Md.)) or publicly available (e.g., G. P .S. Raghava, Biotech Software & Internet Report, 2001, 2: 198-200).

Analysis of tile Fragment Size Distribution of Amniotic Fluid Fetal DNA

The analyzing step in the methods of the invention can be performed using any of a variety of techniques including those described above. In the practice of the present invention, these techniques as well as other techniques known in the art may be used as described or may be modified such that they allow for fragmentation size patterns to be obtained.

A fragment size distribution pattern generally includes one or more of: total number of nucleic acid fragments present in the sample being analyzed, size of one or more nucleic acid fragments in the sample, absolute or relative abundance levels of nucleic acid fragments of a specific size or size range, and absolute or relative abundance levels of nucleic acid fragments of different sizes in the sample. A fragment size distribution pattern may be presented as a graphical representation (e.g., on paper or a computer screen), a physical representation (e.g., a gel or array) or a digital representation stored in a computer-readable medium (e.g., CD, DVD, hard disk drive, magnetic tape or server for streaming media over networks).

Test and Reference Samples

In certain embodiments of the invention, the fragment size pattern of a test sample of amniotic fluid fetal DNA is compared to that of a reference sample of genomic DNA.

Preferably, amniotic fluid fetal DNA is isolated from a sample of amniotic fluid as described above. A test sample of amniotic fluid fetal DNA to be used in the methods of the invention includes a plurality of nucleic acid fragments comprising a substantially complete first genome, whose karyotype is unknown.

A reference sample of control genomic DNA to be used in the methods of the invention includes a plurality of nucleic acid fragments comprising a substantially complete second genome, whose karyotype is known. Genomic control DNA may be selected to act as a negative control (e.g., sample with a normal or wild-type genome) or as a positive control (e.g., sample containing one or more chromosomal aberrations). The reference sample of control DNA may originate from a fetus with either a normal 46, XX karyotype (female euploid) or a normal 46, XY karyotype (male euploid). Alternatively, the reference sample of control genomic DNA may originate from a fetus with an identified chromosomal abnormality (for example, a fetus with trisomy 21). The reference sample of control fetal DNA is preferably isolated from amniotic fluid using the same method as that used for the test sample. The karyotype of the control DNA may be determined by conventional G-banding analysis, metaphase CGH, FISH or SKY (D. W. Bianchi et al., Prenatal. Diagn. 1993, 13: 293-300; D. Ganshirt-Ahlert et al., Am. J. Reprod. Immunol. 1993, 30: 2-3; J. L. Simpson et al., J. Am. Med. Assoc. 1993, 270: 2357-2361; Y. I. Zheng et al., J. Med. Genet. 1993, 30: 1051-1056).

The test and control fetal DNA samples are each submitted to fragment size distribution analysis according to the present invention and their fragment size distribution patterns are compared. As will be recognized by one skilled in the art, the fragment size distribution pattern of the test sample may be compared to more than one control fragment size distribution pattern. For example, the fragment size pattern of the test sample may be compared to fragment size patterns of a karyotypically normal fetus and to fragment size patterns of fetuses with different known chromosomal abnormalities.

Fragment Size Distribution Maps

Information on amniotic fluid fetal DNA fragment size distribution obtained for fetuses with a specific chromosomal abnormality may be grouped to form a fragment size distribution map characteristic for the chromosomal abnormality. Preferably, such fragment size information is obtained as described herein for a statistically significant number of fetuses with the same chromosomal abnormality.

The fragment size distribution map represents a signature or fingerprint for the chromosomal abnormality and provides a template for comparison to fragment size patterns generated from fetuses with unknown karyotype. Fragment size distribution maps may be presented as a graphical representation (e.g., on paper or computer screen), a physical representation (e.g., a gel or array) or a digital representation stored in a computer-readable medium).

III. Prenatal Diagnosis

Practicing the methods of the present invention includes providing a prenatal diagnosis. In certain embodiments, the prenatal diagnosis is provided based on the fragment size pattern of the cell-free fetal DNA isolated from amniotic fluid.

Chromosomal Abnormalities and Associated Diseases and Conditions

Chromosomal aberrations that can be detected and identified by the methods of the present invention include numerical and structural chromosomal abnormalities.

For example, the methods of the invention allow for detection of numerical abnormalities, such as those in which there is an extra set(s) of the normal (or haploid) number of chromosomes (triploidy and tetraploidy), those with a missing individual chromosome (monosomy) and those with an extra individual chromosome (trisomy and double trisomy). The presence of an abnormal number of chromosomes in an otherwise diploid organism is called aneuploidy (see, A. C. Chandley, in: “Human Genetics—Part B: Medical Aspects”, 1982, Alan R. Liss: New York, N.Y.). Approximately half of spontaneous abortions are associated with the presence of an abnormal number of chromosomes in the karyotype of the fetus (M. A. Abruzzo and T. J. Hassold, Environ. Mol. Mutagen. 1995, 25: 38-47), which makes aneuploidy the leading cause of miscarriage. Trisomy is the most frequent type of aneuploidy and occurs in 4% of all clinically recognized pregnancies (T. J. Hassold and P. A. Jacobs, Ann. Rev. Genet. 1984, 18: 69-97). The most common trisomies involve the chromosomes 21 (associated with Down syndrome), 18 (Edward syndrome) and 13 (Patau syndrome) (see, for example, G. E. Moore et al., Eur. J. Hum. Genet. 2000, 8: 223-228). Other aneuploidies are associated with Turner syndrome (presence of a single X chromosome), Klinefelter syndrome (characterized by an XXY karyotype) and XYY disease (characterized by an XYY karyotype).

Fragment size distribution analysis of amniotic fluid fetal DNA according to the methods of the present invention may be used to detect numerical chromosomal abnormalities and therefore to diagnose diseases and conditions associated with aneuploidies including, but not limited to: Down syndrome, Edward syndrome and Patau syndrome, as well as Turner syndrome, Klinefelter syndrome and XYY disease.

Other types of chromosomal abnormalities that can be detected and identified by the methods of the present invention are structural chromosomal aberrations. In contrast to numerical chromosomal abnormalities that correspond to gains or losses of entire chromosomes, structural chromosomal aberrations involve portions of chromosomes. Structural chromosomal aberrations include: deletions (e.g., absence of one or more nucleotides normally present in a gene sequence, absence of an entire gene, or missing portion of a chromosome), additions (e.g., presence of one or more nucleotides normally absent in a gene sequence, presence of extra copies of genes (also called duplications), or presence of an extra portion of a chromosome), rings, breaks, and chromosomal rearrangements, such as translocations and inversions.

The methods of the invention may be used to detect chromosomal abnormalities involving the X chromosome. A large number of these chromosomal abnormalities are known to be associated with a group of diseases and conditions collectively termed X-linked disorders. X-linked disorders include, but are not limited to, Hemophilia A, Duchenne muscular dystrophy, Lesh-Nyhan syndrome, and Fragile X syndrome.

Prenatal Diagnosis

In certain embodiments, the methods of the invention are performed when the pregnant woman is 35 or older. The most common factor associated with high risk outcome of pregnancy is advanced maternal age. In women over the age of 35, the risk of chromosomal abnormality (1% or higher) presumably exceeds the risk of amniocentesis, which explains that more than 90% of amniocenteses are performed on women of advanced maternal age. Yet it has been estimated that up to 80% of Down syndrome infants are born to women under age 35 (L. B. Holmes, New Eng. J. Med. 1978, 298: 1419-1421), who are generally not considered candidates for amniocentesis. This situation has persuaded some investigators to suggest extending the availability of amniocentesis to all women who ask for such a prenatal test.

In other embodiments, the methods of the invention are performed when the fetus carried by the pregnant woman is suspected of having a chromosomal abnormality or when the fetus is suspected of having a disease or condition associated with a chromosomal abnormality. For example, such situations may arise when a previous child of the couple of prospective parents has a chromosomal abnormality, when there is a case of parental chromosomal rearrangement, when there is a case of family history of late-onset disorders with genetic components, when a maternal serum screening test comes back positive, documenting, for example, an increased risk of fetal neural tube defects and/or fetal chromosomal abnormality, or in case of an abnormal fetal ultrasound examination, for example, one that revealed signs known to be associated with aneuploidy.

IV. Kits

In another aspect, the present invention provides kits comprising materials useful for carrying out the methods of the invention. The diagnostic procedures described herein may be performed by diagnostic laboratories, experimental laboratories, or practitioners. The invention provides kits which can be used in these different settings.

Basic materials and reagents required for prenatal diagnosis according to the present invention may be assembled together in a kit. In certain embodiments, the kit comprises one or more of: materials to extract cell-free fetal DNA from amniotic fluid, reagents to perform a fragment size distribution analysis, and instructions for using the kit according to a method of the invention. Each kit necessarily comprises the reagents which render the procedure specific (i.e., kits intended to be used with gel electrophoresis will contain reagents useful to perform gel electrophoresis). Depending on the procedure, the kit may further comprise one or more of: amplification buffer and/or reagents, labeling buffer and/or reagents, and detection means. Protocols for using these buffers and reagents for performing different steps of the procedure may also be included in the kit.

The reagents may be supplied in a solid (e.g., lyophilized) or liquid form. The kits of the present invention optionally comprise different containers (e.g., vial, ampoule, test tube, flask or bottle) for each individual buffer and/or reagent. Each component will generally be suitable as aliquoted in its respective container or provided in a concentrated form. Other containers suitable for conducting certain steps for the disclosed methods may also be provided. The individual containers of the kit are preferably maintained in close confinement for commercial sale.

In certain embodiments, the kits of the present invention further comprise control samples. For example, a kit may include frozen samples of amniotic fluid from fetuses with known karyotypes. In other embodiments, the inventive kits comprise at least one fragment size distribution map as described herein for use as comparison template. For example, a kit may comprise a fragment size distribution map established for karyotypically normal fetuses and a plurality of fragment size distribution maps, each characteristic of a different chromosomal abnormality. Each fragment size distribution profile map may be presented in the form of a graph. Preferably, a fragment size distribution map is digital information stored in a computer-readable medium.

Instructions for using the kit according to a method of the present invention may comprise instructions for extracting fetal DNA from amniotic fluid supernatant samples, instructions for performing the fragment size distribution analysis, instructions for interpreting the results as well as a notice in the form prescribed by a governmental agency (e.g., FDA) regulating the manufacture, use or sale of pharmaceuticals or biological products.

### EXAMPLES

The following examples describe some of the preferred modes of making and practicing the present invention. However, it should be understood that these examples are for illustrative purposes only and are not meant to limit the scope of the invention. Furthermore, unless the description in an Example is presented in the past tense, the text, like the rest of the specification, is not intended to suggest that experiments were actually performed or data were actually obtained.

Most of the experimental results presented below have been described by the Applicants in recent scientific publications (P. B. Larrabee et al., Am. J. Hum. Genet., 2004, 75: 485-491; O. Lapaire et al., Clin. Chem., 2006, 52: 156-157 and O. Lapaire et al., “Cell-Free Fetal DNA in Amniotic Fluid: Unique Fragmentation Signatures in Euploid and Aneuploid Fetuses”, which was submitted for publication, each of which is incorporated herein by reference in its entirety). While working on some of studies reported in these publications, one of the Applicants, Olav Lapaire received a grant from the Swiss National Fund (PBBSB- 108590).

### Example 1

### Amniotic Fluid Fetal DNA Isolation and Preliminary Tests

Frozen amniotic fluid supernatant specimens (38) were obtained from the Tufts-New England Medical Center (Tufts-NEMC) Cytogenetics Laboratory (D. W. Bianchi et al., Clin. Chem. 2001, 47: 1867-1869). All samples were collected for routine indications, such as advanced maternal age, abnormal maternal serum screening results, or detection of a fetal sonographic abnormality. The standard protocol in the Cytogenetics Laboratory is to centrifuge the amniotic fluid sample upon receipt, place the cell pellet into tissue culture, assay an aliquot of the fluid for alpha-fetoprotein and acetyl cholinesterase levels, and store the remainder at −20° C. as a back-up in case of assay failure. After six months, the frozen amniotic fluid supernatant samples are normally discarded.

The frozen fluid samples obtained from the Cytogenetics Laboratory were initially thawed at 37° C. and then mixed with a vortex for 15 seconds. An aliquot of 500 μL of fluid was spun at 14,000 rpm in a microcentrifuge to remove any remaining cells. A final volume of 400 μL of the supernatant was used for extraction of DNA using the “Blood and Body Fluid” protocol as described by Qiagen.

Real-time quantitative PCR analysis was performed using a Perkin-Elmer Applied Biosystems (PE-ABI) 7700 Sequence Detector. Analysis was based on the 5′-to-3′ exonuclease activity of the Tap DNA polymerase, using the FCY locus as a basis for detecting male DNA if the fetus was male. The FCY primers were derived from the Y-chromosome-specific sequence Y49a (DYSI) (G. Lucotte et al., Mol. Cell. Probes, 1991, 5: 359-363). The FCY amplification system consisted of the following amplification primers: FCY-F (5′-TCCTGCTTATCCAAATTCACCAT-3′) and a dual-labeled fluorescent TaqMan probe, FCY-T: (5′-FAMAAGTCGCCACTGGATATCAGTTCCCTTCTTAMRA-3′). The β-globin gene was used to confirm the presence of DNA and estimate its overall concentration.

Amplification reactions were set up as described previously by Y. M .D. Lo et al. (Am. J. Hum. Genet. 1998, 62: 768-775, which is incorporated herein by reference in its entirety), except that each primer was used at 100 nM and the probe was used at 50 nM. Amplification data were collected by the 7700 Sequence Detector and analyzed using the Sequence Detection System software, Ver. 1.6.3 (PE-ABI). Each sample was run in quadruplicate with the mean results of the four reactions used for further calculations. An amplification calibration curve was created using titrated purified male DNA. The extractions and subsequent quantitative assays were performed twice for each sample, with the mean of the two results used for final analysis.

In 21 samples, the known fetal karyotype was 46, XX (normal female), in 15 samples the known fetal karyotype was 46, XY (normal male), and in two samples, the known karyotype was 47, XY, +21 (male fetus with Down syndrome). However, the samples were coded and analyzed blindly. The mean amount of β-globin DNA detected was 3,427 GE/mL (range 293-15,786). There was no correlation between gestational age and the total amount of DNA detected. In the female fetuses 0 GE/mL of DYSI DNA was detected in the amniotic fluid. The mean value of DYSI DNA detected in male fetuses was 2,668 GE/mL (range 228-12,663 GE/mL). Linear regression analysis showed a correlation between fetal DNA and gestational age (r=0.6225, p=0.0231). In all 38 cases, the predicted fetal gender was correct. The results were statistically significant (p<0.0001, by Fisher's exact test). In the cases of fetal Down syndrome, there was no elevation of the amount of fetal DNA compared to the samples obtained from fetuses with a normal male karyotype.

These data show that there is 100-200 fold more fetal DNA per milliliter of fluid in the amniotic fluid compartment, as compared with maternal serum and plasma. Therefore, amniotic fluid appears as a previously unappreciated rich source of fetal nucleic acids that can be obtained relatively easily by using standard procedures.

### Example 2

### Improvements in Amniotic Fluid Fetal DNA Isolation Method

Using the “Blood and Body Fluid” vacuum protocol, only a minor proportion of amniotic fluid supernatant samples could be further analyzed (e.g., with genomic microarrays, in which a minimum of 100 ng of DNA is necessary) (P. B. Larrabee et al., Am. J. Hum. Genet., 2004, 75: 485-491).

As reported above, the original method was based on known protocols for the extraction of cell-free fetal DNA from maternal plasma/serum, as specific guidelines for the extraction of DNA from amniotic fluid did not exist. Therefore, further investigation was needed to optimize cell-free fetal DNA extraction from amniotic fluid supernatant to more fully exploit this promising source of genetic material.

Approval for this study was obtained from the institutional review Board of Tufts-New England Medical Center (Boston, Mass.) and Women and Infants Hospital (Providence, R.I.). For protocol optimization, five large volume samples of amniotic fluid supernatant were obtained from patients undergoing therapeutic amnioreduction for twin-twin syndrome (TTS). Once the protocol was optimized, comparison of the DNA yield between old and new protocols was made using freshly discarded amniotic fluid supernatant samples from 29 euploid singleton pregnancies. The median gestational age at amniocentesis was 16.9 weeks (25^{th} and 75^{th} percentiles: 16.4 and 18.1 weeks).

To improve the yield of extracted cell-free fetal DNA, the original method (P. B. Larrabee et al., Am. J. Hum. Genet., 2004, 75: 485-491) using the “Blood and Body Fluid” vacuum protocol (Qiagen, Valencia, Calif.) was modified in different ways as described below. (1) The vacuum extraction pressure was increased to 800 mbar to allow for maximal absorption of DNA to filters. Although this pressure may exceed that available in some laboratories (e.g., using building vacuum pressure), reduced vacuum leads to lower yield of extracted DNA. (2) the volume over-loaded Mini Spin Columns were replaced by Maxi Spin Columns (Qiagen). In the original protocol, 10 mL of amniotic fluid was added to 1 mL of protease, 10 mL of AL lysis buffer (Qiagen) and 10 mL of 100% ethanol, which exceeded the volumetric capacity of the mini columns. The use of maxi columns allows for large starting volumes to be processed and therefore a larger quantity of cell-free fetal DNA can be obtained as compared to mini columns. (3) AL buffer was substituting with AVL buffer (Qiagen). AL buffer used in the original extraction protocol, was selected based on prior experience for the isolation of cell-free fetal DNA from plasma and serum, as there was no information available on the most suitable buffer for extraction of cell-free fetal DNA from amniotic fluid. However, AVL buffer supplemented with DNA carrier for the extraction of low concentrations of target DNA was selected for the current protocol on the basis of similar qualities between amniotic fluid and urine, a bodily fluid in which AVL buffer is recommended for DNA extraction.

Real-time quantitative PCR analysis was performed in triplicate for each sample using a 7700 Sequence Detector (Perkin-Elmer Applied Biosystems (PE-ABI) Forster City, Calif.), with the mean result of the three reactions used for further calculations. Amplification of the glyceraldehyde-3-phosphate dehydrogenase (GAPDH) locus was performed to determine the quantity and quality of total DNA in amniotic fluid supernatant as previously described (K. L. Johnson et al., Clin. Chem., 2004, 50: 516-521). Reactions were set up in a 50 μL volume, using 25 μL of PE-ABI Universal Mastermix and 5 μL of extracted DNA. Primers and probes were used at a final concentration of 300 and 200 nM, respectively. Data were analyzed using the Sequence Detection System Software, version 1.6.3 (PE-ABI). Two samples with no template DNA were included on each reaction plate as negative controls. Cycling conditions for all reactions consisted of a 2 minute incubation at 50° C. to allow for UNGerase activity, an initial denaturation step at 95° C. for 10 minutes, and then 40 cycles at 95° C. for 15 seconds and 60° C. for 1 minute. The results were expressed as genome equivalents per milliliter (GE/mL) using a conversion factor of 6.6 pg of DNA per cell, taking into account the elution and starting volumes (Y. M. Lo et al., Am. J. Hum. Genet., 1998, 62: 768-775).

Large volume samples of amniotic fluid supernatant (n=5) were used for initial experiments to determine the effects of changes in columns, lysis buffer, and high vacuum pressure (800 mbar) on DNA yield. High vacuum pressure was used for all experiments with Mini Spin columns; appropriate centrifugation speed was used for DNA isolation with Maxi Spin columns. The use of mini columns and AL buffer (i.e., the original protocol) led to a fetal DNA yield of 224 GE/mL from one amniotic fluid sample; further assessment of this procedure was not performed due to the low yield obtained. Substituting AL with AVL buffer (using mini columns) led to a mean fetal DNA yield of 1470.16 GE/mL (SD=455.59), and replacing mini columns with maxi columns (using AL buffer) led to a mean fetal DNA yield of 1563.60 GE/mL (SD=623.39). Finally, substituting AL with AVL buffer and replacing mini columns with maxi columns led to a mean DNA yield of 1972.04 GE/mL (SD=786.08).

The improved protocol using maxi columns and AVL buffer was then tested to determine if traditional DNA extraction, i.e., phenol, chloroform and isoamyl alcohol, (“Molecular Cloning. A Laboratory Manual”, L. Sambrook et al. (Eds.), 1989, 2^{nd} Ed., Cold Spring Harbor Laboratory Press) further improved yield. This change resulted in a decreased yield from 5648 to 1121 GE/mL in one large volume sample. Further assessment of this extraction method was not performed due to the low DNA yield obtained.

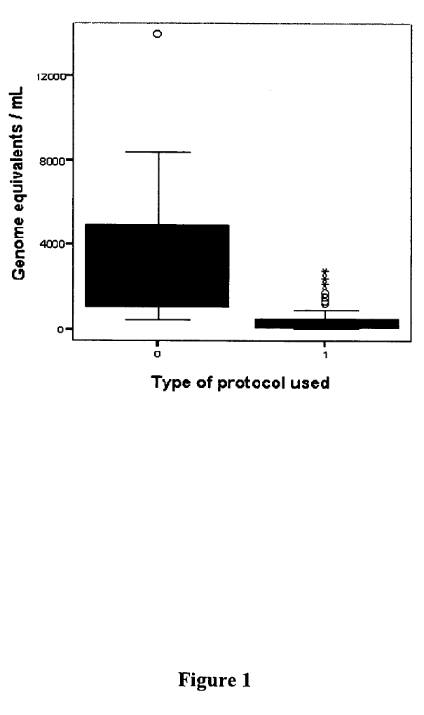

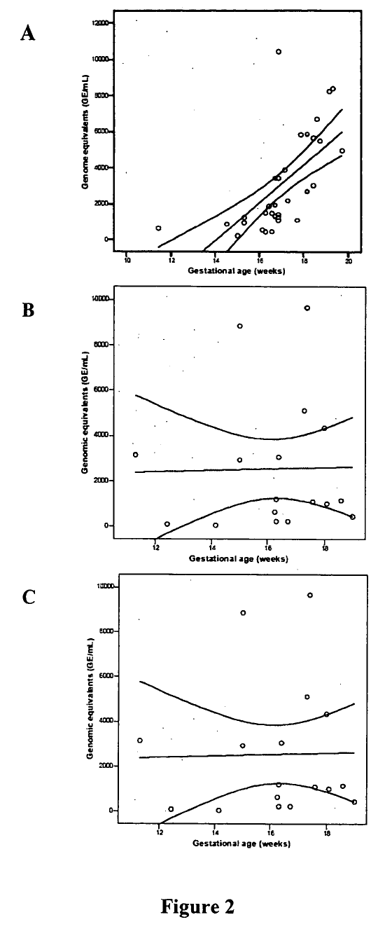

From euploid singleton pregnancies (n=29), the median amount of GAPDH DNA extracted from 10 mL of amniotic fluid with the new protocol was 1700 GE/mL (25^{th}, 75^{th} percentiles: 1071, 4938 GE/mL, respectively) compared to 246 GE/mL (25^{th}, 75^{th} percentiles: 93, 523.5 GE/mL) using the original protocol (P. B. Larrabee et al., Am. J. Hum. Genet., 2004, 75: 485-491) (p<0.0001; Wilcoxon signed rank test) (FIG. 1). The proportion of samples that had sufficient yield of extracted DNA for subsequent chromosome microarray analysis (i.e. ≧100 ng) also increased compared to the original protocol, from 39% (28 of 72) (P. B. Larrabee et al., Am. J. Hum. Genet., 2004, 75: 485-491) to 86% (25 of 29) (p<0.0001, χ² test).

Several advantages have been realized with the protocol developed here. In addition to an improved yield from a greater proportion of samples as compared to the original protocol, the new protocol allows for the extraction of cell-free fetal DNA from up to 10 samples in less than three hours. The replacement of AL buffer with AVL buffer eliminates the need for a heating bath during the lysis step, and fewer overall steps are involved in the protocol (which decreases the chance of potential contamination). However, the cost of cell-free fetal DNA extraction from a 10 mL AF supernatant sample using the new protocol is about 10 fold higher compared to the original protocol (about $39 and about $4 per sample, respectively), although the advantage of the new protocol with respect to improved DNA yield justifies this higher cost per sample.

For clinical applications, one advantage of using the amniotic fluid supernatant is its availability without interfering with current standard of care or compromising fetal health. Another advantage is the ability to freeze the supernatant sample at −80° C. without risking a significant degradation of DNA over time, thus allowing for the batch processing of multiple samples (T. Lee et al., Am. J. Obstet. Gynecol., 2002, 187: 1217-1221). For research applications, the development of an optimized protocol will allow for further investigation of the origin and kinetics of cell-free fetal DNA. It has been suggested that placental abnormalities and pregnancy-associated disorders may affect cell-free fetal DNA levels in the maternal serum (X. Y. Zhong et al., Am. J. Obstet. Gynecol., 2001, 184: 414-419; D. W. Swinkels et al., Clin. Chem., 2002, 48: 650-653; T. W. Lau et al., Clin. Chem., 2002, 48: 2141-2146; R. J. Levine et al., Am. J. Obset. Gynecol., 2004, 190: 707-713), whereas fetal organs that come in contact with amniotic fluid (such as lungs, kidneys, and the gastrointestinal system) and fetal disorders may affect cell-free fetal DNA levels in amniotic fluid.

In conclusion, the improvements to the original protocol for the extraction of cell-l free fetal DNA from amniotic fluid supernatant resulting in statistically significantly higher yields of high quality cell-free fetal DNA, allowing for a substantial majority of samples to be analyzed with subsequent molecular methods (e.g., genomic microarrays) to further assess for sub-microscopic abnormalities that are associated with specific clinical findings. The improvements demonstrated here make it possible to augment current standard of care (i.e., the metaphase karyotype) through the analysis of this previously unappreciated source of fetal nucleic acids. Furthermore, the improved yield of cell-free fetal DNA will allow for exploration of the currently unknown genetic, pathophysiological and kinetic issues of cell-free fetal DNA in amniotic fluid.

### example 3

### Investigation of Amniotic Fluid Fetal DNA Fragmentation Pattern

To date, no study has addressed the biochemical properties of cell-free fetal DNA in amniotic fluid. This is in contrast to maternal plasma, in which it has been shown that circulating fetal DNA sequences are smaller than maternal-derived ones, on the order of less than 300 base pairs (Y. Li et al., Clin. Chem., 2004, 50: 1002-1011; K. C. A. Chan et al., Clin. 2004, 50: 88-92). This distinct property has been used to increase the yield of fetal DNA extracted from maternal samples to permit non-invasive prenatal diagnosis of β-thalassemia (Y Li et al., JAMA, 2005, 292: 843-849).

The Applicants have hypothesized that cell-free fetal DNA in amniotic fluid would have different biophysical properties that cell-free fetal DNA in maternal plasma. Since second trimester amniotic fluid is composed predominantly of fetal urine, the Applicants speculated that passage of cell-free fetal DNA through the fetal kidneys might affect its qualities. Additional variables such as karyotype, gestational age, and storage at −80° C. were also examined.

Material and Methods

DNA Extraction from 10 mL of Amniotic Fluid. Approval for this study was from the institutional review boards of Tufts-New England Medical Center and Women and Infants Hospital. Ten (10) mL of residual fresh AF supernatant, taken for clinical indications, were obtained from women carrying euploid fetuses (n=39) and aneuploid fetuses (n=4). To test the effects of storage and karyotype, samples frozen at −80° C. were obtained from euploid fetuses (n=19) and from aneuploid fetuses with trisomies 21 (n=16), 18 (n=9), 13 (n=3) triploidy (n=4), and monosomy X (n=2) (see Table 1). DNA extraction was performed using the QIAamp DNA Maxi Kit (Qiagen, Valencia, Calif.) in combination with a 40 mL of AVL buffer (Qiagen), supplemented with nucleic acid carrier, and 40 mL of 100% ethanol as previously described (Lapaire et al., Clin. Chem., 2006, 52: 156-157). Final elution was performed with 2 mL of AE buffer (Qiagen). The extracted DNA was stored at −80° C. until further processing. Before storage, the purity of the eluted DNA was assessed with a Biophotometer (Eppendorf, Hamburg, Germany).

To measure the amount of the extracted cell-free fetal DNA, real-time quantitative PCR analysis was performed in triplicate using the 7700 Sequence Detector (Applied Biosystems, Foster City, Calif.), with the mean result of the three reactions used for further calculations. Amplification of the housekeeping gene glyceraldehyde-3-phosphate dehydrogenase (GAPDH) locus was performed on cell-free fetal DNA in amniotic fluid supernatant as described previously (K. L. Johnson et al., Clin. Chem., 2004, 50: 516-521). Reactions were set up in a 50 μL volume, using 25 μL of Universal Mastermix (Applied Biosystems) and 5 μL of extracted DNA. Primers and probes were used at a final concentration of 300 and 200 nM, respectively. Data were analyzed using the Sequence Detection System software, version 1.6.3 (Applied Biosystems). Two samples with no template DNA were included on each reaction plate as negative controls. Cycling conditions for all reactions consisted of a 2 minute incubation at 50° C. to allow for UNGerase activity, an initial denaturation step of 95° for 10 minutes, and then 40 cycles at 95° C. for 15 seconds and 60° C. for 1 minute. The results were expressed as genome equivalents per milliliter (GE/mL) using a conversion factor of 6.6 pg of DNA per cell (Y. M. Lo et al., Am. J. Hum. Genet., 1998, 62: 768-775)

DNA Electrophoresis and Staining. Standard methods were used for the preparation of the 1% agarose gels, using 1 ×TAE buffer (40 mM Tris acetate, 2 mM Na₂EDTA2H₂O, pH 8.5). Twenty (20) μL of the eluted, non amplified cell-free fetal DNA was added and thoroughly mixed with 5 μL of loading buffer (Blue Juice, Invitrogen, Grand Island, N.Y.), consisting of 65% (w/v) sucrose, 10 mM Tris-HCI (pH 7.5), 10 mM EDTA, and 0.3% (w/v) bromophenol blue, which co-migrates with ˜0.5 kb DNA fragments. Double-stranded cell-free fetal DNA from each sample was separated by electrophoresis in two parallel electrophoresis systems (Owi Separation Systems, Portsmouth, N.H.). The gels, 7-8 mm thick, were run by step-wise increasing in voltage throughout electrophoresis for better resolution from 2.9 V/cm for 60 minutes, followed by 5.9 V/cm for 60 minutes, up to 8.75 V/cm for 35 minutes. For fragment size estimation, a 1 KB extension ladder (Invitrogen) was used. The ladder consisted of 8 bands containing multiples of a 1018 bp DNA fragment, vector bands of 506/517 bp, 1636 bp and additional bands of 5, 10, 20 and 40 kb. After electrophoresis, the gels were incubated for 20 minutes in SYBR Gold staining solution (Invitrogen), diluted 1: 10,000 fold in 1×TAE buffer, with gentle rocking.

Gel Imaging and Data Analysis. Photographic images were taken while trans-illuminating the gel at 300 nm (Ultra Lum, model UVB-10, Carson, Calif.) using a camera (Polaroid Model QSP) with an exposure time of 1 second, aperture of 4.5, and film designed for capturing high quality electrophoresis images (Polaroid 667 Film ISO 3000/DIN 36). The images were saved as Tagged Image File Format (.tif) after scanning with a ScanJet 6300c using PrecisionScan Pro software (Hewlett Packard, Palo Alto, Calif.) and transferred to GeneTool software (Syngene, Frederick, Md.). After importing the .tif files into GeneTool, the tracks on the gel were analyzed automatically. For calibration, data from the 1 KB extension ladder were used. Data were created by repeatedly measuring the sum of the pixel values along the band representing each sample (i.e., raw volume). The number of measurements for each sample ranged between 515 and 536. The gel running distance was expressed as retention factor (Rf) distance, which is equivalent to relative mobility. Relative mobility is defined as the distance migrated by a band divided by the distance migrated by the dye front. The Rf values lie between 0 and 1, with lower Rf values representing larger DNA fragments.

Statistical analyses. Descriptive statistics, including medians, 25^{th} and 75^{th} percentile ranges, were generated for all study variables. The non-parametric Kruskal-Wallis test was used to compare unadjusted GAPDH levels between trisomy 18, trisomy 21 and euploid pregnancies. Spearman correlation analysis was carried out between GAPDH levels and gestational age. Due to the small sample sizes of the other aneuploid samples (trisomy 13 [n=3], triploidy [n=4], and monosomy X [n=2]), the separate statistical analysis was not performed, although the descriptive characteristics were provided.

The effect of interaction between the karyotype and gestational age on the logarithmically transformed GAPDH levels was assessed using multiple linear regression analyses. All statistical analyses were performed using SAS/STAT software (SAS Institute, Inc., Cary, N.C.). Statistical significance was assigned where P value was less than 0.05

Statistical analysis for fragmentation signature. Fragmentation signature was analyzed using the trapezoid methods. Area under the curve (AUC) was calculated for each sample separately using all available signal readings. Log-transformed total AUC and AUC for different DNA molecular weights (i.e. distances run by half of the cell-free fetal DNA fragments through the gel) were compared between frozen euploid and aneuploid samples, as well as fresh and frozen euploid samples, using linear regression analysis after adjustment for the initial amount of PCR product and gestational age. Correlation between AUC and the initial PCR product was assessed using Spearman correlation analysis simultaneously controlling for GA.

Results

Real-time PCR analysis using GAPDH locus was carried out on cell-free fetal DNA extracted from all 96 amniotic fluid samples. Furthermore, the fragment size distribution of cell-free fetal DNA from 51 of 96 samples were analyzed after gel electrophoresis and staining.

Fresh Euploid Amniotic Fluid Samples

The data showed that the concentration of cell-free fetal DNA from fresh euploid amniotic fluid samples correlates significantly with gestational age (R²=−0.77, p<0.0001). Median amounts of cell-free fetal DNA from fresh amniotic fluid samples are presented in Table 1.

Fresh vs. Frozen Euploid Samples

Data from 19 frozen amniotic fluid samples from euploid singleton fetuses suggested a statistically significant influence of storage time. The median amount of cell-free fetal DNA in frozen euploid samples, measured by GAPDH, was significantly lower than the median amount in fresh euploid samples (p<0.0001, adjusted for gestational age) (see Table 1). However, no linear relationship was seen between storage time and levels of cell-free fetal DNA in frozen euploid samples (p=0.19).

In contrast to fresh euploid samples, in which gestational age is a statistically significant predictor of cell-free fetal DNA levels (p<0.0001), cell-free fetal DNA levels in frozen samples were not statistically associated with gestational age (p=0.63). However, a significant storage time-gestational age interaction was observed (p=0.02).

Euploid vs. Aneuploid Euploid Samples

Compared to frozen euploid samples, a statistically significant decrease in the median amount of cell-free fetal DNA was observed in the sub-groups of frozen aneuploid sample, when adjusted for gestational age (p=0.0005).

The concentration of cell-free fetal DNA from aneuploid samples correlated marginally with gestational age in all combined aneuploid samples (R²=0.32, p=0.08). Statistically significant correlations were not seen in trisomy 21 (R²=0.32, p=0.93) and trisomy 18 (R 2=0.04, p=0.94), although this lack of correlation may be due to their small sample sizes.

A small number of fresh aneuploid samples were analyzed (n=4), which included one trisomy 21, one triploidy and two monosomy X samples. While the small number precludes statistical analyses for each aneuploidy type, the median amount of cell-free fetal DNA in fresh aneuploid samples was 2.3 times higher than that of frozen aneuploid samples (4600 vs. 1714 GE/mL). This difference is consistent with that seen in euploid samples (i.e., 2.6 times higher in fresh—1424 GE/mL—versus frozen samples—606 GE/mL).

Fragmentation Signature-Qualitative and Quantitative Analysis

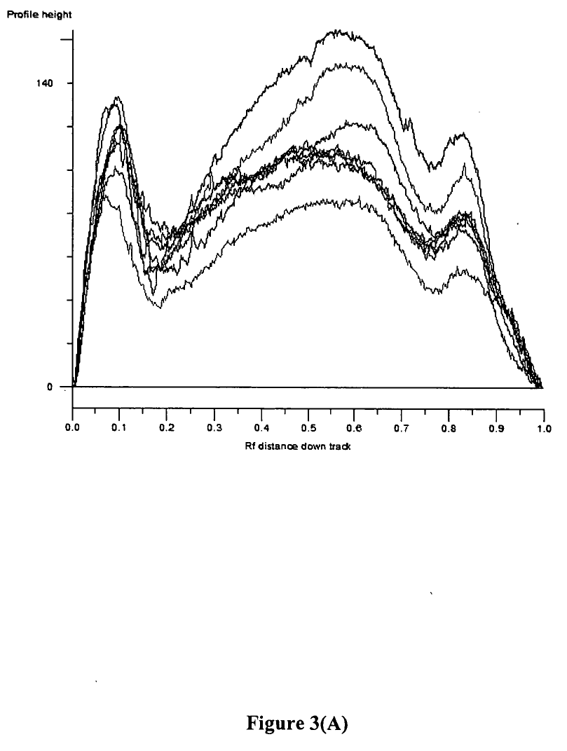

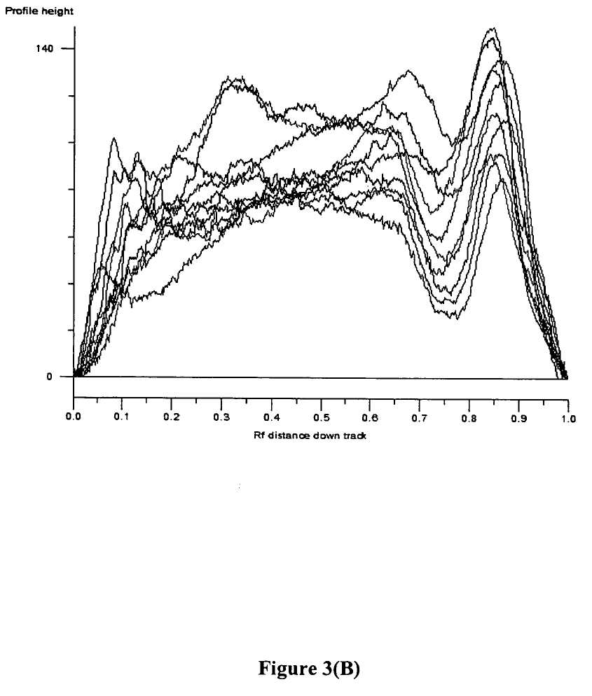

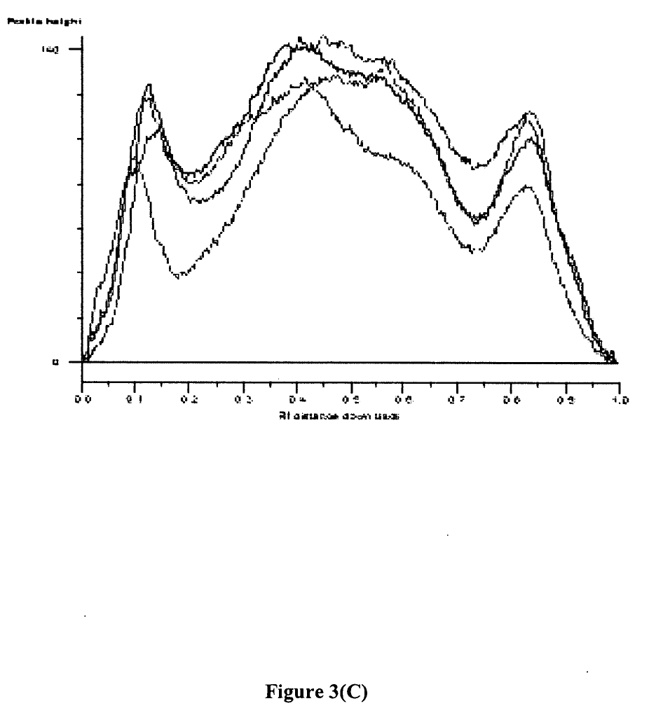

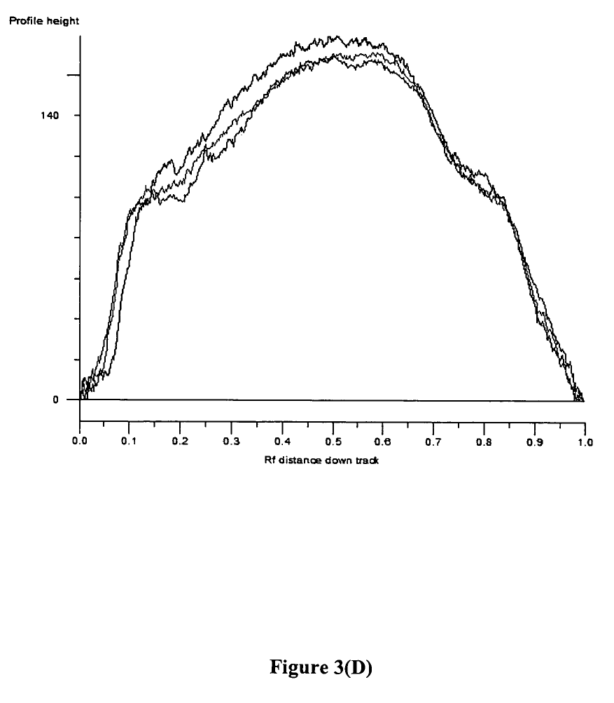

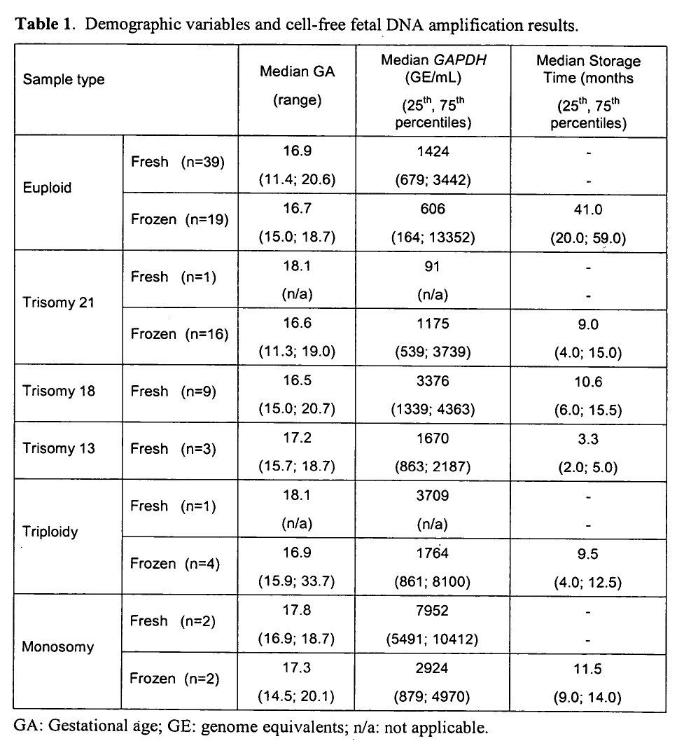

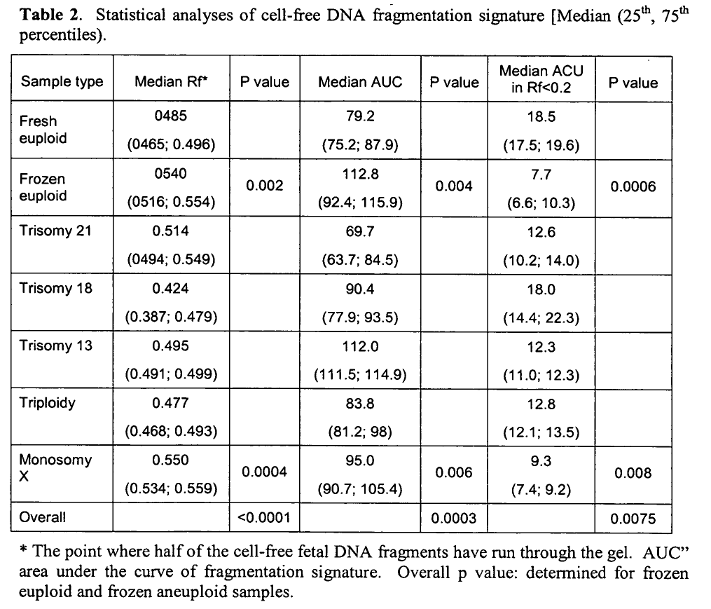

Following gel electrophoresis, scanning and software analysis, unique qualitative patterns were observed for euploid and each aneuploid that were termed “fragmentation signatures” (see FIG. 3(A-D)). For each karyotype group these patterns were remarkably consistent in different individual samples.

To perform quantitative analysis, a measurement was developed in which the discriminative fragmentation signatures of fresh and frozen euploid and aneuploid samples were expressed by the distance (Rf) where half of the cell-free fetal DNA fragments have run through the gel. There were significant differences in this measurement between fresh euploid and frozen euploid amniotic fluid samples (p=0.0002) and among all frozen aneuploid amniotic fluid samples (p=0.0004) (see Table 2).

The median AUC for DNA fragments of different lengths was determined for fresh and frozen euploid amniotic fluid samples as well as for aneuploid amniotic fluid samples. Statistical analysis showed highly significant differences in AUC among fresh and frozen euploid samples and aneuploid samples, when adjusted for the initial cell-free fetal DNA amount, as estimated by real-time quantitative PCR analysis using GADPH (overall p=0.0003) (Table 2). The results remained statistically significant after additional adjustment for gestational age.

Fresh euploid samples showed significantly higher molecular weight cell-free fetal DNA fragments than frozen aneuploid and euploid samples. This was determined by analyzing the median percentage of the estimated amount of cell-free fetal DNA that ran in the first fifth of the gel running distance (Rf<0.2). These results are shown in Table 2. In addition to a significant overall difference in this measure along all amniotic fluid samples (p=0.0075), a significant loss of large fragments was observed in the frozen euploid samples compared to fresh euploid samples (p=0.0006, unadjusted for gestational age).

Discussion

In this study, significant differences in the quantitative levels of amniotic fluid cell-free fetal DNA were observed as a function of gestational age, karyotype and sample storage time. However, the most intriguing finding of the present study is the novel fragmentation signature pattern of amniotic fluid cell-free fetal DNA. Striking differences were observed in cell-free fetal DNA fragment sizes and their characteristic distributions as a function of karyotype and sample storage. The Applicants hypothesized that these differences may be due to either the different sources of the cell-free fetal DNA (i.e., fetal organs that come in contact with amniotic fluid such as lungs, kidneys, dermis, and the gastrointestinal system) or differences in DNA metabolism that are affected by karyotype.

The present results show that there is a unique and consistent qualitative pattern of amniotic fluid cell-free fetal DNA fragments in euploid and aneuploid fetuses. The fragmentation signature, which can be demonstrated rapidly at low cost on standard agarose gels, represents differences in the proportions of different sizes of cell-free fetal DNA fragments, and suggests specific pathognomonic kinetic mechanisms. The results may have clinical applications in the rapid triaging of amniotic fluid. Furthermore, the ability to statistically analyze the data from each sample provides a novel tool for a predictive model of aneuploidy in prenatal diagnosis.

The specific fragmentation signatures may be explained by different apoptotic pathways and/or variable activation of the necrotic pathway. DNA degradation is considered to be one of the defining hallmarks of apoptosis. Apoptotic fragmentation is commonly a two-step process in which DNA is first cleaved into fragments of 50-300 kilobases, termed high molecular weight (HMW) DNA fragmentation. Subsequently, DNA is cleaved between nucleosomes in smaller fragments of oligonucleosomal size, also described as low molecular weight (LMW) DNA ladder (H. Lecoeur, Exp. Cell Res., 2002, 277: 1-14).

Fresh euploid amniotic fluid showed a significant higher percentage of larger DNA fragments than frozen euploid samples, whereas aneuploid samples, like trisomy 21, featured smaller fragments, irrespective of sample storage time. The Applicants hypothesized that, as in cancer cell lines, in which an asynchronous apoptotic process leads to a decrease in fragment size (R. Oberhammer et al., EMBO J., 1993, 12: 3679-3684), the same mechanism can explain the observed differences between the euploid and aneuploid samples. Furthermore, the activation of cysteine-dependent aspartate-specific proteases (known as caspases) by upstream pathways, triggered by the underlying karyotype, may initiate apoptosis or enzymatically cleave cellular components.

Up- or down-regulation of genes involved in apoptosis may play an important role in trisomy 21 and may affect detectable cell-free fetal DNA levels. ETS2, a member of the ET family of transcription factors, which have been proposed to have important functions in immune responses, cancer and bone development, is located on chromosome 21 (21p22.3) (N. Sacchi et al., Science, 1986, 231: 379-382). This gene is over-expressed in brains and fibroblasts of individuals with trisomy 21. Over-expression in some of the trisomy 21 samples may lead to an increase of the p53 dependent apoptosis pathway, as seen in prior studies (E. J. Wolvetang et al., Hum. Mol. Genet., 2003, 12: 247-255). On the other hand, alternative forms of cell-free fetal DNA release, like necrosis, may also contribute to the varied and gestational-age independent levels of amniotic fluid cell-free fetal DNA in aneuploid fetuses. The distinction between apoptosis and necrosis is not always well defined, and in many instances these two models may be regarded as a continuum of cell death.

Other pathways, like necrosis or active secretion, may also contribute to the excretion of cell-free fetal DNA. Evidence suggests that in the case of aneuploidy, non-physiologic cell death as a result of primary stress signals or secondary to apoptosis (J. Savill et al., Nat. Rev. Immunol., 2002, 2: 965-975) contributes in greater proportion to the release of cell-free fetal DNA than in euploid fetuses. Therefore, this mode of cell-free fetal DNA release may contribute substantially to different fragmentation signatures and cell-free fetal DNA levels in abnormal karyotypes.

Specific pathologic processes occurring in fetal organs that are in direct contact with amniotic fluid may also affect the fragment distribution of cell-free fetal DNA. Interestingly, two distinct fragmentation signatures were observed in the trisomy 18 samples. This may be explained by differences in the extent of renal dysplasia, a common feature of trisomy 19.

Sample stability during storage at −80° C. is an important variable in basic and clinical research, which often relies on archived samples. Prior to this study, there was no data about the effect of storage of cell-free fetal DNA in amniotic fluid. Cell-free fetal DNA in maternal plasma is reported to be stable at −20° C. for more than 4 years (K. Koide et al., Prenat. Diagn., 2005, 25: 604-607). The Applicants have previously demonstrated a storage-related decline in cell-free fetal DNA concentration in maternal plasma of −0.66 GE/mL per month (T. Lee et al., Am. J. Obstet. Gynecol., 2002, 187: 1217-1221). The present results show that storage of amniotic fluid, even at −80° C., significantly decreases the yield and the integrity of cell-free fetal DNA. No linear relationship was seen between storage time and levels of cell-free fetal DNA, suggesting a more rapid degradation of the non-particle-associated form of cell-free fetal DNA, than the particle associated form (P. Larrabee et al., Clin. Chem., 2005, 51: 1024-1026)

In conclusion, the present data suggest that gestational age, karyotype, and sample storage time affect quantitative levels of cell-free fetal DNA, as well as cell-free fetal DNA fragment size in amniotic fluid; this may be due to fundamental differences in tissue sources, excretion modes and/or kinetic pathways in direct contact with amniotic fluid. Characteristics patterns, unique for each common aneuploidy, may offer the possibility of using DNA fragmentation analysis as a rapid and cost-effective means of triaging amniotic fluid samples.

### OTHER EMBODIMENTS

Other embodiments of the invention will be apparent to those skilled in the art from a consideration of the specification or practice of the invention disclosed herein. It is intended that the specification and examples be considered as exemplary only, with the true scope of the invention being indicated by the following claims.

What is claimed is:

1. A method of prenatal diagnosis comprising steps of:
providing a sample of amniotic fluid fetal DNA comprising a plurality of fetal DNA fragments having various sizes;
analyzing the amniotic fluid fetal DNA to obtain a fragment size distribution pattern of the amniotic fluid fetal DNA; and
based on the fragment size distribution pattern obtained, providing a prenatal diagnosis.

2. The method of claim 1, wherein the amniotic fluid fetal DNA is obtained by:
providing a sample of amniotic fluid obtained from a woman pregnant with a fetus;
removing cell populations from the sample of amniotic fluid to obtain a remaining amniotic fluid material; and
treating the remaining amniotic material such that cell-free fetal DNA present in the remaining amniotic material is extracted and made available for analysis, resulting in amniotic fluid fetal DNA.

3. The method of claim 2, wherein substantially all cell populations are removed from the sample of amniotic fluid and wherein the amniotic fluid fetal DNA consists essentially of cell-free fetal DNA.

4. The method of claim 2, wherein the remaining amniotic material comprises some cell populations and wherein the amniotic fluid fetal DNA comprises cell-free fetal DNA and DNA originating from the cells present in the remaining amniotic material.

5. The method of claim 2 further comprising steps of:
freezing the remaining amniotic material to obtain a frozen sample;
storing the frozen sample for a period of time under suitable storage conditions; and
thawing the frozen sample prior to the treating step.

6. The method of claim 5 further comprising removing substantially all cell populations that are still present in the remaining amniotic material after the thawing step and prior to the treating step.

7. The method of claim 2, wherein analyzing the amniotic fluid fetal DNA to obtain a fragment size distribution pattern comprises submitting the amniotic fluid fetal DNA to a gel electrophoresis, capillary gel electrophoresis, flow cytometry or MALDI-TOF mass spectrometry analysis.

8. The method of claim 7, wherein analyzing the amniotic fluid fetal DNA to obtain a fragment size distribution pattern comprises submitting the amniotic fluid fetal DNA to a gel electrophoresis analysis.

9. The method of claim 2, wherein providing a prenatal diagnosis comprises one or more of: detecting a chromosomal abnormality, identifying a chromosomal abnormality, and identifying a disease or condition associated with a chromosomal abnormality affecting the fetus.

10. The method of claim 2, wherein the fetus is suspected of having a disease or condition associated with a chromosomal abnormality

11. The method of claim 10, wherein the disease or condition associated with a chromosomal abnormality is an aneuploidy.

12. The method of claim 11, wherein the aneuploidy is selected form the group consisting of Down syndrome, Patau syndrome, Edward syndrome, Turner syndrome, Klinefelter syndrome, and XYY disease.

13. The method of claim 2, wherein the pregnant woman is 35 or more than 35 years old.

14. The method of claim 2 further comprising comparing the fragment size distribution pattern obtained to at least one fragment size distribution pattern obtained for a sample of control amniotic fluid fetal DNA, prior to providing a prenatal diagnosis

15. The method of claim 14, wherein the control amniotic fluid fetal DNA is from a karyotypically and developmentally normal fetus.

16. The method of claim 14, wherein the control amniotic fluid fetal DNA is from a fetus with an identified chromosomal abnormality.

17. The method of claim 2 further comprising steps of:
repeating all the previous steps for a statistically significant number of amniotic fluid fetal DNA samples from karyotypically and developmentally normal fetuses; and
using the fragment size distribution patterns obtained to establish a fragment size distribution map for amniotic fluid fetal DNA from karyotypically and developmentally normal fetuses.

18. The method of claim 2 further comprising steps of:
repeating all the previous steps for a statistically significant number of amniotic fluid fetal DNA samples from fetuses with an identical chromosomal abnormality; and
using the fragment size distribution patterns obtained to establish a fragment size distribution map for amniotic fluid fetal DNA from fetuses with that particular chromosomal abnormality.

19. The method of claim 2 further comprising comparing the fragment size distribution pattern obtained to at least one fragment size distribution map prior to providing a prenatal diagnosis.

20. The method of claim 19 wherein the fragment size distribution map is characteristic of a normal karyotype.

21. The method of claim 19, wherein the fragment size distribution map is characteristic of a chromosomal abnormality.

22. A kit for prenatal diagnosis comprising one or more of: materials to extract fetal DNA from a sample of amniotic fluid obtained from a pregnant woman;
materials to analyze amniotic fluid fetal DNA to obtain a fragment size distribution pattern;
at least one fragment size distribution map; and
instructions for using the kit for providing prenatal diagnosis as set forth in claim 19.


SEQUENCE LISTING (2 sequences)

SEQ ID NO: 1 - DNA, 23 bp, Artificial
  Amplification primer
tcctgcttatccaaattcaccat

SEQ ID NO: 2 - protein, 37 aa, Artificial
  Amplification primer
FAMAAGTCGCCACTGGATATCAGTTCCCTTCTTAMRA